(19) United States
(12) Patent Application Publication
(10) Pub. No.: US 2026/0083777 A1
(43) Pub. Date: Mar. 26, 2026

(54) CITRULLINATED ANTIGEN-SPECIFIC CHIMERIC ANTIGEN RECEPTORS FOR TARGETING REGULATORY T CELLS TO TREAT HIDRADENITIS SUPPURATIVA

(71) Applicant: Sonoma Biotherapeutics, Inc., South San Francisco, CA (US)

(72) Inventors: Leonard DRAGONE, San Francisco, CA (US); Joshua BEILKE, Seattle, WA (US)

(73) Assignee: Sonoma Biotherapeutics, Inc., South San Francisco, CA (US)

(21) Appl. No.: 19/112,106

(22) PCT Filed: Sep. 18, 2023
(86) PCT No.: PCT/US2023/074509

§ 371 Date: Mar. 14, 2025

Related U.S. Application Data
(60) Provisional application No. 63/408,046, filed on Sep. 19, 2022.
(60) Provisional application No. 63/519,184, filed on Aug. 11, 2023.

(51) Int. Cl. A61K 35/17; A61K 40/11; A61K 40/31; A61K 40/41; C07K 14/535; C07K 14/705; C07K 14/725; C07K 16/18; C12N 5/0783

(52) U.S. Cl. CPC A61K 35/17; A61K 40/11; A61K 40/31; A61K 40/416; C07K 14/535; C07K 14/7051; C07K 14/70517; C07K 14/70521; C07K 16/18; C12N 5/0637; A61K 2239/13; A61K 2239/17; A61K 2239/21; C07K 2317/53; C07K 2317/565; C07K 2317/622; C07K 2319/02; C07K 2319/03; C12N 2510/00

(57) ABSTRACT

Disclosed herein are chimeric antigen receptors (CARs) comprising an antigen binding site that recognizes citrullinated polypeptides. Citrullinated polypeptides, such as citrullinated vimentin, are expressed in the skin lesions of subjects with hidradenitis suppurativa. Further disclosed are T cells, and in particular, Treg cells that express these CARs. Administration of these CAR-T cells is useful in the treatment of hidradenitis suppurativa.

### CROSS REFERENCE TO RELATED APPLICATIONS

This application claims the benefit of U.S. Provisional Application No. 63/519,184, filed Aug. 11, 2023, and U.S. Provisional Application No. 63/408,046, filed Sep. 19, 2022, each of which is hereby incorporated by reference in its entirety.

### REFERENCE TO AN ELECTRONIC SEQUENCE LISTING

The contents of the electronic sequence listing (237752000740seqlist.xml; Size: 66,037 bytes; and Date of Creation: Sep. 13, 2023) are herein incorporated by reference in their entirety.

### FIELD

The present disclosure relates to chimeric antigen receptors reactive with citrullinated antigens and regulatory T cells expressing the receptors for treating hidradenitis suppurativa.

### BACKGROUND

Hidradenitis suppurativa (HS) is a chronic inflammatory skin condition that manifests in the development of painful skin lesions formed through follicular occlusion caused by hyperkeratinization and hyperplasia of the infundibular epithelium. Disease progression eventually leads to ‘plugging’ and rupture of the hair follicle unit, which initiates a chronic cutaneous inflammatory response. HS has a profound negative effect on patients due to disease-associated pain and movement restrictions, the malodor and drainage from affected skin, and disfigurement that accompanies the HS symptoms. Current treatment options are insufficient in providing symptom resolution and there remains an unmet need for effective therapies.

Regulatory T (Treg) cells have been found to be enriched in HS skin lesions, yet there is an imbalance in the ratio of Treg to CD4+ helper T17 (Th17) cells in favor of Th17. Normalization of this ratio has the potential to ameliorate immune dysregulation in HS. Pre-clinical studies have shown that antigen specific and autologous Treg cells have the potential to treat systemic inflammation and organ injury supporting the hypothesis that Treg cells could lead to transformative and durable treatments. Recent studies have shown the therapeutic potential of Treg-related immune therapies, including low dose interleukin-2 (IL-2) and IL-2 muteins, in a variety of autoimmune settings. Clinical studies have also evaluated the potential of polyclonal Treg cells as a treatment option for organ transplant recipients and patients suffering from autoimmune conditions. Although the Treg cell infusions were well tolerated, only limited efficacy was observed.

Currently there is no cure for HS, as well as for many other chronic inflammatory diseases. This is due in part to the fact that the cause of HS in not known. In fact, HS is frequently treated with corticosteroids or other anti-inflammatory agents that may leave recipients more susceptible to contracting infectious diseases. Thus, what is needed in the art are treatments that target antigens preferentially found in HS lesions.

### SUMMARY

The present disclosure relates to chimeric antigen receptors reactive with citrullinated antigens and regulatory T cells expressing the receptors for treating hidradenitis suppurativa. In particular, the CARs expressed by Treg cells of the present disclosure specifically bind to citrullinated polypeptides, including vimentin, citrullinated filaggrin and citrullinated fibrinogen, and citrullinated fragments thereof. In some embodiments, the single chain fragment variable (scFv) part of the CAR was obtained from an antibody highly specific for one or more citrullinated proteins. In one embodiment, specific scFv chains were inserted into a second-generation CAR construct. In some embodiments, the scFv chains were inserted into a CAR construct cloned into a lentiviral vector. In the detailed description, references to antibodies are applicable to antigen-binding domains of the CARs of the present disclosure unless the context indicates otherwise.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1A: Citrullinated protein (CitP) is frequently present in samples of hidradenitis suppurativa (HS) lesions, but not in samples of normal skin (non-HS). FIG. 1B: CitP is also frequently present in synovial tissue samples from rheumatoid arthritis (RA) patents, but not in synovial tissue samples from healthy subjects (non-RA). CitP reactivity was measured by detecting binding of an anti-citrullinated vimentin (CV) antibody (e.g., anti-CV Ab referred to herein as S01-mIgG2a).

FIG. 2A-2C: HS skin lesions have higher levels of inflammation and CitP target antigen compared to normal skin. FIG. 2A: HS skin lesions have a higher percentage of inflammatory cells than normal skin. FIG. 2B: HS skin lesions have greater numbers of CitP+ inflammatory cells than normal skin. FIG. 2C: HS skin lesions also have greater numbers of CitP+ non-inflammatory cells than normal skin.

FIG. 3: An exemplary CV-reactive chimeric antigen receptor (CAR) expressed on the surface of a reporter cell line is activated by citrullinated vimentin, fibrinogen, and two different filaggrin proteins but not by non-citrullinated vimentin, fibrinogen, and filaggrin proteins.

FIG. 4A-4B: Higher levels of CitP are present in serum from patients with HS and RA than in serum from normal control subjects as determined by measuring binding of an anti-CV Ab (S01-mIgG2a) in a cell based assay and an ELISA, respectively. FIG. 4C: Higher levels of citrullinated and MMP-degraded vimentin (VICM) are present in serum from patients with HS and RA than in serum from normal control subjects.

FIG. 5A-5B: Serum samples of HS patients have elevated levels of C-reactive protein (CRP) and anti-citrullinated protein antibodies (ACPA) as compared to normal serum samples.

FIG. 6A: A schematic overview of a cell-based assay for detecting CitP. FIG. 6B: Serum samples of HS patients have elevated levels of citrullinated-antigen (CitP) compared to samples of normal serum samples.

FIG. 7A-7D: HS patients have a comparable Treg frequency and phenotype as healthy donors (HD).

FIG. 8A-8B: Treg frequency and recovery are comparable for HS patients (HS, Hurley 2, and Hurley 3) and HD during production of CV-CAR Treg cells. FIG. 8C: CV-CAR Treg cells derived from PBMC of HD and HS patients (HS, Hurley 2, and Hurley 3) express comparable levels of FOXP3. Hurley 2 and Hurley 3 represent patients with moderate and severe HS, respectively.

FIG. 9A-9B: CV-CAR Treg cells derived from PBMC of HD or HS patients proliferate and are similarly activated by CAR stimulation (pCV beads).

FIG. 10A-10B: Treg cells derived from PBMC of HD or HS patients exhibit similar suppressive activity.

### DETAILED DESCRIPTION

Citrullination of proteins is an irreversible posttranslational modification that impacts protein structure and is implicated in the regulation of histones, the cytoskeleton, and function of secreted proteins. In addition, citrullinated proteins accumulate at sites of chronic inflammation. Citrulline is generated via a post-translation conversion of arginine to citrulline by peptidylarginine deiminase (PAD) enzymes. It has been shown that PAD enzymes are strongly expressed in activated myeloid cells, including macrophages and neutrophils. PAD enzymes play an important role in formation of neutrophil extracellular trap (NETs), a phenomenon that externalizes autoantigens and immunostimulatory molecules. During NETosis, neutrophils externalize citrullinated autoantigens, releasing DAMPs as innate immune activators, which are implicated in driving HS pathogenesis. While expressed intracellularly, aberrant PAD activity can lead to the deposition of citrullinated proteins in many tissues, including the joints, lungs, lymph nodes, and periodontal tissues in patients with inflammatory diseases. The activity of PAD enzymes is increased in the inflamed skin of HS patients, particularly within neutrophils, which release citrullinated proteins and other autoantigens during NETosis. Citrullinated vimentin (CV) and fibrinogen are found in the extracellular matrix of inflamed skin of hidradenitis suppurativa (HS) patients. Citrullination also leads to the generation of autoantigens during inflammatory responses. Citrullinated autoantigens are highly immunogenic, can be presented on MHC molecules, and induce T-cell-mediated B cell activation. These lead to the development of pathogenic anti-citrullinated protein antibodies (ACPAs), which have been observed in serum and at the sites of skin lesions in HS patients. The ACPAs can react with several citrullinated proteins including citrullinated fibrinogen, myeloperoxidase, nucleosome, histone H4, and dsDNA. Taken together, the presence of NETs, citrullinated proteins, and ACPAs in HS skin lesions indicates a pathogenic role for citrullination in HS disease etiology.

The present disclosure describes a targeted antigen-specific approach to increase the functionality and quantity of Treg cells that can be activated in sites of disease, such as HS lesions. Targeting citrullinated proteins with CAR-Treg cells as described herein, is believed to be a new approach for treatment of HS.

### I. DEFINITIONS

Unless otherwise specified, terms and symbols of biochemistry, nucleic acid chemistry, molecular biology, developmental biology and molecular genetics follow those of standard treaties and texts in the field, for example, Sambrook et al, Molecular Cloning: A Laboratory Manual, 2nd Edition (Cold Spring Harbor Press, 1989); Alberts and Singer, Developmental Biology, Eighth Edition (Sinauer Associates Inc., Sunderland, MA, 2006); Kornberg and Baker, DNA Replication, Second Edition (W.H. Freeman, New York, 1992); Gaits, ed., Oligonucleotide Synthesis: A Practical Approach (IRL Press, Oxford, 1984); Lehninger, Biochemistry, Second Edition (Worth Publishers, New York, 1975); Eckstein, ed., Oligonucleotides and Analogs: A Practical Approach (Oxford University Press, New York, 1991); and the like.

As used herein, the terms “antigen,” “immunogen,” and “antibody target,” refer to a molecule, compound, or complex that is recognized by an antibody, i.e., can be bound by the antibody. The term can refer to any molecule that can be recognized by an antibody, e.g., a polypeptide, polynucleotide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

As used herein, the term “epitope” refers to the localized site on an antigen that is recognized and bound by an antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope).

As used herein, the term “antibody” refers to a polypeptide comprising a framework region from an immunoglobulin gene, that specifically bind and recognize an antigen. Typically, the “variable region” contains the antigen-binding region of the antibody (or its functional equivalent) and is most critical in specificity and affinity of binding. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD).

Antibodies can be of (i) any of the five major classes of immunoglobulins, based on the identity of their heavy-chain constant domains—alpha (IgA), delta (IgD), epsilon (IgE), gamma (IgG) and mu (IgM), or (ii) subclasses (isotypes) thereof (E.g., IgG1, IgG2, IgG3, IgG4, IgA1 and IgA2). The light chains can be either lambda or kappa.

The following are a non-exhaustive list of different antibody forms, all retaining antigen binding activity:
(1) whole immunoglobulins (also referred to as “intact” antibodies) (two light chains and two heavy chains, e.g., a tetramer);
(2) an immunoglobulin polypeptide (a light chain or a heavy chain);
(3) an antibody fragment, such as Fv (a monovalent or bi-valent variable region fragment, and can encompass only the variable regions (e.g., V_{L} and/or V_{H}), Fab (V_{L}C_{L} V_{H}C_{H}), F(ab′)2, Fv (V_{L}V_{H}), scFv (single chain Fv) (a polypeptide comprising a V_{L} and V_{H} joined by a linker, e.g., a peptide linker), (scFv)2, sc(Fv)2, bispecific sc(Fv)2, bispecific (scFv)2, minibody (sc(FV)2 fused to CH3 domain), diabody (noncovalent dimer of single-chain Fv (scFv) fragment that consists of the heavy chain variable (VH) and light chain variable (VL) regions connected by a small peptide linker), triabody is trivalent sc(Fv)3 or trispecific sc(Fv)3;
(4) a multivalent antibody (an antibody comprising binding regions that bind two different epitopes or proteins, e.g., “scorpion” antibody;
(5) a fusion protein comprising a binding portion of an immunoglobulin fused to another amino acid sequence (such as a fluorescent protein); and
(6) heavy chain only antibody or antibody fragment having only two heavy chains and lacking the two light chains usually found in antibodies.

Production and properties of tandem scFvs and diabodies are described, e.g., in Asano et al. (2011) J Biol. Chem. 286:1812; Kenanova et al. (2010) Prot Eng Design Sel 23:789; Asano et al. (2008) Prot Eng Design Sel 21:597.

The phrase “CDR sequence set” as used herein refers to the 3 heavy chain and/or 3 light chain CDRs of a particular antibody described herein. A “light chain” CDR sequence set refers to the light chain CDR sequences. A “heavy chain” CDR sequence set refers to the heavy chain CDR sequences. A “full” CDR sequence set refers to both heavy chain and light chain CDR sequences. CDRs are predicted based on IMGT sequence alignment.

As used herein, the term “chimeric antibody” refers to an antibody having amino acid sequences derived from two or more species. In one embodiment, the variable region of both light and heavy chains correspond to the variable region of antibodies derived from one species of mammal (e.g., mouse, rat, rabbit, etc.) with the desired specificity, affinity and capability, while the constant region are homologous the sequence derived from another species (typically in the subject receiving the therapy, e.g., human) to avoid eliciting an immune response.

As used herein, the term “humanized antibody” refers to a chimeric antibody in which the CDRs, obtained from the VH and VL regions of a non-human antibody having the desired specificity, affinity and capability are grafted to a human framework sequence. In one embodiment, the framework residues of the humanized antibody are modified to refine and optimize the antibody specificity, affinity and capability. Humanization, i.e., substitution of non-human CDR sequences for the corresponding sequences of a human antibody, can be performed following the methods described in, e.g., U.S. Pat. Nos. 5,545,806; 5,569,825; 5,633,425; 5,661,016; Riechmann et al., Nature 332:323-327 (1988); Marks et al., Bio/Technology 10:779-783 (1992); Morrison, Nature 368:812-13 (1994); Fishwild et al., Nature Biotechnology 14:845-51 (1996).

As used herein, the term “human antibody” refers to an antibody produced by a human or an antibody having an amino acid sequence corresponding thereto made by any technique known in the art.

The specificity of the binding can be defined in terms of the comparative dissociation constants (Kd) of the antibody (or other targeting moiety) for target, as compared to the dissociation constant with respect to the antibody and other materials in the environment or unrelated molecules in general. A larger (higher) Kd is a Kd that describes a lower affinity interaction. Conversely a smaller (lower) Kd is a Kd that describes a higher affinity interaction or tighter binding. By way of example only, the Kd for an antibody specifically binding to a target may be femtomolar, picomolar, nanomolar, or micromolar and the Kd for the antibody binding to unrelated material may be millimolar or higher. Binding affinity can be in the micromolar range (kD=10⁻⁴ to 10⁻⁶), nanomole range (kD=10⁻⁷ M to 10⁻⁹ M), picomole range (kD=10⁻¹¹ M to 10⁻¹² M), or femtomole range (kD=10⁻¹³ M to 10⁻¹⁵ M).

As used herein, an antibody “binds” or “recognizes” an antigen or epitope if it binds the antigen or epitope with a Kd of less than 10⁻⁴M (i.e., in the micromolar range). The term “binds” with respect to a cell type (e.g., an antibody that binds cells expressing an antigen), typically indicates that an agent binds a majority of the cells in a pure population of those cells. For example, an antibody that binds a given cell type typically binds to at least ⅔ of the cells in a population of the indicated cells (e.g., 67, 75, 80, 85, 90, 91, 92, 93, 94, 95, 96, 97, 98, 99, or 100%). In some cases, binding to a polypeptide can be assayed by comparing binding of the antibody to a cell that presents the polypeptide to binding (or lack thereof) of the antibody to a cell that does not express the polypeptide. One of skill will recognize that some variability will arise depending on the method and/or threshold of determining binding. Affinity of an antibody for a target can be determined according to methods known in the art, e.g., as reviewed in Ernst et al. Determination of Equilibrium Dissociation Constants, Therapeutic Monoclonal Antibodies (Wiley & Sons ed. 2009).

As used herein, the term “greater affinity” as used herein refers to a relative degree of antibody binding where an antibody X binds to target Y more strongly (Kon) and/or with a smaller dissociation constant (Koff) than to target Z, and in this context antibody X has a greater affinity for target Y than for Z. Likewise, the term “lesser affinity” herein refers to a degree of antibody binding where an antibody X binds to target Y less strongly and/or with a larger dissociation constant than to target Z, and in this context antibody X has a lesser affinity for target Y than for Z. The affinity of binding between an antibody and its target antigen, can be expressed as KA equal to 1/KD where KD is equal to kon/koff. The kon and koff values can be measured using surface plasmon resonance technology, for example, using a Molecular Affinity Screening System (MASS-1) (Sierra Sensors GmbH, Hamburg, Germany). An antagonist or blocking antibody is an antibody that partially or fully blocks inhibits or neutralizes a biological activity related to the target antigen relative to the activity under similar physiological conditions when the antibody is not present. Antagonists can be competitive, non-competitive or irreversible. A competitive antagonist is a substance that binds to a natural ligand or receptor at the same site as the natural ligand-receptor interaction or binds allosterically in a manner that induces a change to prevent normal binding. A non-competitive antagonist binds at a different site than the natural ligand-receptor interaction, but lower the KD or signal resulting from the interaction. An irreversible inhibitor causes covalent modifications to the receptor preventing any subsequent binding.

As used herein, the term “avidity” refers to the overall stability of the binding complex between the antibody and the target antigen. It is governed by three factors, (i) the intrinsic affinity of the antibody for the antigen, (2) the valency of the antibody, and (3) the geometric arrangement of the interacting components. Affinity is the strength of the interaction between the antibody and a single target, whereas avidity is an accumulated strength of multiple affinities. In one embodiment, the antibodies provided herein are divalent.

As used herein, an antibody “preferentially binds” binds a first antigen relative to a second antigen if it binds the first antigen with greater affinity than it does the second antigen. Preferential binding can be at least any of 2-fold, 5-fold, 9-fold, 10-fold, 20-fold, 30-fold, 40-fold, 50-fold, 100-fold, 500-fold or 1000-fold greater affinity.

As used herein, an antibody “specifically binds” or is “specific for” a target antigen or target group of antigens if it binds the target antigen or each member of the target group of antigens with an affinity of at least any of 1×10⁻⁶ M, 1×10⁻⁷M, 1×10⁻⁸ M, 1×10⁻⁹ M, 1×10⁻⁰ M, 1×10⁻¹¹ M, 1×10⁻¹² M, and, for example, binds to the target antigen or each member of the target group of antigens with an affinity that is at least two-fold greater than its affinity for non-target antigens to which it is being compared. Typically, specific binding is characterized by binding the antigen with sufficient affinity that the antibody is useful as a diagnostic to detect the antigen or epitope and/or as a therapeutic agent in targeting the antigen or epitope.

As used herein, the term “polypeptide” refers to a molecule having a sequence of natural and/or unnatural amino acids connected through peptide bonds. The term “peptide” refers to a short polypeptide, typically no more than 30 amino acids long. The amino acid sequence of a polypeptide is referred to as its “primary structure.” The term “protein” refers to a polypeptide having a secondary, tertiary and/or quaternary structure, e.g., structures stabilized by hydrogen bonds, relationships between secondary structures and structures formed of more than one protein. Proteins can be further modified by other attached moieties such as carbohydrate (glycoproteins), lipids (lipoproteins) phosphate groups (phosphoproteins) and the like.

As used herein, an amino acid sequence “consists of” only the amino acids in that sequence.

As used herein, a first amino acid sequence “consists essentially of” a second amino acid sequence if the first amino acid sequence (1) comprises the second amino sequence and
(2) is no more than 1, no more than 2 or no more than 3 amino acids longer than the second amino acid sequence.

As used herein, a first amino acid sequence is a “fragment” of a second amino acid sequence if the second amino acid sequence comprises the first amino acid sequence. In certain embodiments, a first amino acid sequence that is a fragment of a second amino acid sequence may have no more than any of 1, 2, 3, 4, 5, 6, 7, 8, 9 or 10 fewer amino acids than the second amino acid sequence.

As used herein, a “functional equivalent” of a reference amino acid sequence is a sequence that is not identical to the reference sequence, but that contains minor alterations such as, for example, insertion, deletion or substitution of one or a few amino acids. A functionally equivalent sequence retains the function (e.g., immunogenicity) of the reference sequence to which it is equivalent. If a functionally equivalent amino acid sequence contains substitution of one or more amino acids with respect to the reference sequence, these will generally be conservative amino acid substitutions.

As used herein, a “conservative amino acid substitution” is one in which one amino acid residue is replaced with another amino acid residue without abolishing the protein's desired properties. Suitable conservative amino acid substitutions can be made by substituting amino acids with similar hydrophobicity, polarity, and R-chain length for one another. See, e.g., Watson, et al., “Molecular Biology of the Gene,” 4th Edition, 1987, The Benjamin/Cummings Pub. Co., Menlo Park, CA, p. 224. Examples of conservative amino acid substitution include the following (Note, some categories are not mutually exclusive):

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic (non-polar, | Ala, Val, Ile, Leu, Met, Gly, Pro |
| hydrophobic) | |
| Basic | Lys, Arg, His |
| Aromatic | Phe, Tyr, Trp |

As used herein, the term “substantially identical” refers to identity between a first amino acid sequence that contains a sufficient or minimum number of amino acid residues that are i) identical to, or ii) conservative substitutions of aligned amino acid residues in a second amino acid sequence such that the first and second amino acid sequences have a common structural domain and/or common functional activity and/or common immunogenicity. For example, amino acid sequences that contain a common structural or antigenic domain having at least about 90%, 91%, 92%, 93%, 94%, 95%, 96%, 97%, 98% or 99% identity are termed sufficiently or substantially identical. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, or encode polypeptides having the same immunogenic properties.

As used herein, a chemical entity, such as a polypeptide, is “substantially pure” or “isolated” if it is the predominant chemical entity of its kind (e.g., of polypeptides) in a composition. This includes the chemical entity representing more than 50%, more than 80%, more than 90%, more than 95%, more than 98%, more than 99%, more than 99.5%, more than 99.9%, or more than 99.99% of the chemical entities of its kind in the composition. A substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules, stabilizers (e.g., BSA), and elemental ion species are not considered macromolecular species for purposes of this definition.

The phrase “isolated antibody” refers to antibody produced in vivo or in vitro that has been removed from the source that produced the antibody, for example, an animal, hybridoma or other cell line (such as recombinant insect, yeast or bacterial cells that produce antibody).

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences maximally aligned by IMGT. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, multiplied by 100 to convert to percentage.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. It is understood that polynucleotides comprising non-transcribable nucleotide bases may be useful as probes in, for example, hybridization assays. The nucleic acid can be either double stranded or single stranded, and represents the sense or antisense strand. Further, the term “nucleic acid” includes the complementary nucleic acid sequences as well as codon optimized or synonymous codon equivalents.

The term “isolated nucleic acid” as used herein refers to a nucleic acid substantially free of cellular material or culture medium when produced by recombinant DNA techniques, or chemical precursors, or other chemicals when chemically synthesized. An isolated nucleic acid is also substantially free of sequences that naturally flank the nucleic acid (i.e., sequences located at the 5′ and 3′ ends of the nucleic acid) from which the nucleic acid is derived.

Hybridization may occur to all or a portion of a nucleic acid sequence molecule. The hybridizing portion is typically at least 15 (e.g., 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(%(G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example, if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: A Laboratory Manual, Cold Spring Harbor Laboratory Press, 2001.

As used herein, the term “expression construct” refers to a polynucleotide comprising an expression control sequence operatively linked with a heterologous nucleotide sequence (i.e., a sequence to which the expression control sequence is not normally connected to in nature) that is to be the subject of expression. As used herein, the term “expression vector” refers to a polynucleotide comprising an expression construct and sequences sufficient for replication in a host cell or insertion into a host chromosome. Plasmids and viruses are examples of expression vectors. As used herein, the term “expression control sequence” refers to a nucleotide sequence that regulates transcription and/or translation of a nucleotide sequence operatively linked thereto. Expression control sequences include promoters, enhancers, repressors (transcription regulatory sequences) and ribosome binding sites (translation regulatory sequences).

As used herein, a nucleotide sequence is “operatively linked” with an expression control sequence when the expression control sequence functions in a cell to regulate transcription of the nucleotide sequence. This includes promoting transcription of the nucleotide sequence through an interaction between a polymerase and a promoter.

The term “vector” as used herein comprises any intermediary vehicle for a nucleic acid molecule which enables said nucleic acid molecule, for example, to be introduced into prokaryotic and/or eukaryotic cells and/or integrated into a genome, and include plasmids, phagemids, bacteriophages or viral vectors such as retroviral based vectors, lentiviral vectors, Adeno Associated viral vectors and the like. The term “plasmid” as used herein generally refers to a construct of extrachromosomal genetic material, usually a circular DNA duplex, which can replicate independently of chromosomal DNA.

“Transfection” refers to the introduction of new genetic material into a cell. It includes transformation (the direct uptake and incorporation of exogenous genetic material from its surroundings through the cell membrane), transduction (the introduction of foreign DNA by a bacteriophage virus into a host cell) and conjugation.

As used herein, a “host cell” refers to a recombinant cell comprising an expression construct.

As used herein, the term “biological sample” refers to a sample containing cells (e.g., cells) or biological molecules derived from cells.

As used herein, the term terms “therapy,” “treatment,” “therapeutic intervention” and “amelioration” refer to any activity resulting in a reduction in the severity of symptoms. The terms “treat” and “prevent” are not intended to be absolute terms. Treatment and prevention can refer to any delay in onset, amelioration of symptoms, improvement in patient survival, increase in survival time or rate, etc. Treatment and prevention can be complete or partial. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques. “Treating” and “Treatment” can also mean prolonging survival as compared to expected survival if not receiving treatment. “Treating” and “treatment” as used herein also include prophylactic treatment.

Compositions or methods “comprising” or “including” one or more recited elements may include other elements not specifically recited (e.g., open-ended terms meaning including but not limited to). For example, a composition that “comprises” or “includes” an antibody may contain the antibody alone or in combination with other ingredients. In contrast, the phrase “consisting of” is closed, indicating that such embodiments do not include additional elements. The term “consisting essentially of” refers to the inclusion of recited elements and other elements that do not materially affect the basic and novel characteristics of a claimed combination (e.g., partially closed term). It is understood that aspects and embodiments described herein as “comprising” include “consisting of” and “consisting essentially of” embodiments.

As used herein, the following meanings apply unless otherwise specified. The word “may” is used in a permissive sense (i.e., meaning having the potential to), rather than the mandatory sense (i.e., meaning must). The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”.

### II. CHIMERIC ANTIGEN RECEPTORS

“Chimeric antigen receptors” or “CARs” are engineered molecules comprising an optional signal peptide, a target binding domain, an optional hinge region, a transmembrane domain, an intracellular signaling domain and an optional co-stimulatory domain. CARs are based on the structure of T cell receptors, which are expressed on T cells and which are involved in the cell-mediated immune response. The “target binding domain” is also referred to herein as an “antigen binding domain”, and as such the term “target” encompasses an “antigen.”

So-called “first-generation” CARs had a targeting domain and a CD3ξ signal transduction domain. So-called “second generation” CARs further included a co-stimulatory domain, such as a CD28 or 4-1 BB domain. So-called “third generation” CARs comprise multiple co-stimulatory domains. So-called “fourth generation” CARs, also referred to as “TRUCKS” are engineered to release a transgenic cytokine upon CAR signaling.

Chimeric antigen receptors (“CARs”) include the following elements: (1) an optional signal peptide, (2) a target binding domain, (3) an optional hinge region; (4) a transmembrane region; (5) an intracellular domain comprising a signal transduction domain. Optionally, the CAR can include any of: a CD3ζ signal transduction domain, an Fc receptor signal transduction domain, a co-stimulatory (signal transduction) domain. That is, these optional elements can be included in addition to or instead of other optional elements. The target binding domain is heterologous to at least one of the other domains. That is, the target binding domain does not naturally occur on a T cell receptor, or is not in the same protein as at least one of the other domains.

The “target binding domain” provides binding specificity to the CAR. The “signal peptide” guides the polypeptide through the cell membrane. The target binding domain can bind to a domain of an antibody that binds to the target antigen for a so-called “Universal CAR”. The “hinge region” is a flexible connector region, e.g., a natural or synthetic polypeptide, or any other type of molecule, providing structural flexibility and spacing to flanking polypeptide regions. The “transmembrane domain” is a membrane-spanning protein domain, typically hydrophobic. The “signal transduction domain” or “signaling domain” transmits a signal through a signal transduction pathway into the cell upon binding. Such signaling activates an activity of the cell. “Co-stimulatory domains” are accessory signaling domains that further transmit signals.

In some embodiments, the CAR comprises:
(i) an target-binding domain (also referred to herein as an antigen binding domain) reactive with a citrullinated protein or citrullinated fragment thereof, such as a VH-VL or VL-VH, wherein the two variable domains are separated by a flexible linker of from 15-25 amino acids in length;
(ii) a hinge domain;
(iii) a co-stimulatory domain; and
(iv) an intracellular signaling domain (also referred to herein as an activation domain). That is, in some embodiments the CAR comprises an antigen-binding domain fused to a CAR platform comprising a hinge domain, a transmembrane domain, and an intracellular domain comprising a costimulatory domain and an activation domain. The CAR may further include a signal peptide (also referred to herein as a leader sequence) to direct expression of the CAR to the surface of a cell, such as a Treg.

In some embodiments, the CAR comprises an antigen-binding domain fused in frame to a CAR platform comprising the amino acid sequences of SEQ ID NO:15, SEQ ID:17, SEQ ID NO:28, and SEQ ID:19. In other embodiments, the CAR comprises an antigen-binding domain fused in frame to a CAR platform comprising the amino acid sequences of SEQ ID NO:30, SEQ ID:16, SEQ ID NO:28, and SEQ ID:19. In some embodiments, the CAR comprises an antigen-binding domain fused in frame to a CAR platform comprising the amino acid sequences of SEQ ID NO:30, SEQ ID:16, SEQ ID NO:29, and SEQ ID:19. In other embodiments, the CAR comprises an antigen-binding domain fused in frame to a CAR platform comprising the amino acid sequences of SEQ ID NO:30, SEQ ID:16, SEQ ID NO:29, and SEQ ID:19.

### A. Signal Peptide

A signal peptide can be any peptide having the function of allowing a polypeptide to traverse a cell membrane. The signal peptide can be derived from CD4, CD8, CD28, TLR or immunoglobulin family of receptors.

For example, the signal peptide can comprise the sequence:

(SEQ ID NO: 18)
MLLLVTSLLLCELPHPAFLLIP;
or
(SEQ ID NO: 23)
MALPVTALLLPLALLLHAAR.

### B. Target Binding Domain

### 1. Structure

The target binding domain can include any polypeptide comprising a target binding function, e.g., an antibody as defined herein. In one embodiment, the target binding domain can comprise an antibody form retaining antigen binding activity as defined herein. In one embodiment, the target binding domain can comprise a single chain antibody (scFV). The scFv can be connected to the transmembrane domain via a hinge domain whose length, flexibility and origin provides variability in the CAR's design, and can, along with the transmembrane domain, contribute to the interaction with antigen, building of the immunologic synapse and impact the CAR's association with additional proteins needed to impart a robust activation signal.

### 2. Targets/Antigens

Chimeric antigen receptors disclosed herein comprise a target binding domain (also referred to herein as an antigen-binding domain) that binds to citrullinated antigens, e.g., those found in the skin lesions of subjects with hidradenitis suppurativa. In particular, the target binding domain can bind to one or more of (i) citrullinated vimentin, (ii) citrullinated filaggrin, (iii) citrullinated fibrinogen and (iv) citrullinated peptides thereof. In some embodiments, the target binding domain can bind to a citrullinated peptide fragment of (i)-(iii), wherein the peptide fragment is at least of 10 amino acids in length, e.g., of at least 12 amino acids, at least 14 amino acids or at least 16 amino acids in length. In some embodiments, the target binding domain further binds to tenascin C. In some embodiments, the target binding domain can bind to two or more of (i) citrullinated vimentin, (ii) citrullinated filaggrin, (iii) citrullinated fibrinogen, and (iv) tenascin C, or citrullinated peptide fragments thereof.

In some embodiments, the target domain is one or more citrullinated peptides selected from the following sequences:

(SEQ ID NO: 24)
ST(Cit)SVSSSSY(Cit)(Cit)MFGG;
(SEQ ID NO: 25)
VYAT(Cit)SSAV(Cit)L(Cit)SSV;
(SEQ ID NO: 26)
(Cit)PAPPPISGGGY(Cit)A(Cit);
(SEQ ID NO: 27)
SHQEST(Cit)GRSRGRSGRSGS.

In some embodiments, the antigen binding domain binds to one or more citrullinated peptides, but does not bind to non-citrullinated counterparts. In some embodiments, the antigen binding domain binds to a citrullinated vimentin peptide set forth as SEQ ID NO:24, but not STRSVSSSSYRRMFGG (SEQ ID NO:45). In some embodiments, the antigen binding domain binds to a citrullinated vimentin peptide set forth as SEQ ID NO:25, but not VYATRSSAVRLRSSV (SEQ ID NO:46). In some embodiments, the antigen binding domain binds to a citrullinated fibrinogen peptide set forth as SEQ ID NO:26, but not RPAPPPISGGGYRAR (SEQ ID NO:47). In some embodiments, the antigen binding domain binds to a citrullinated filaggrin peptide set forth as SEQ ID NO:27, but not SHQESTRGRSRGRSGRSGS (SEQ ID NO:48).

The target binding domain can comprise sequences from an antibody VH and VL domain. In some embodiments, the target binding domain can comprise sequences from heavy chain only antibody or antibody fragment having only two VH domains. This includes particular CDR sets from VH and VL domains. In some embodiments the target binding domain comprises the CDRs from the VH domains of SEQ ID NO:1 or SEQ ID NO:2. In some embodiments the target binding domain comprises the CDRs from the VL domains of SEQ ID NO:3 or SEQ ID NO:4. In some embodiments the target binding domain comprises the CDRs from VH and VL domains of SEQ ID NO:1 and SEQ ID NO:3, respectively. In some embodiments the target binding domain comprises the CDRs from VH and VL domains of SEQ ID NO:1 and SEQ ID NO:4, respectively. In some embodiments the target binding domain comprises the CDRs from VH and VL domains of SEQ ID NO:2 and SEQ ID NO:3, respectively. In some embodiments the target binding domain comprises the CDRs from VH and VL domains of SEQ ID NO:2 and SEQ ID NO:4, respectively.

In some embodiments the target binding domain comprises the complementarity-determining regions (CDRs) from VH and VL domains of SEQ ID NO:1 (S01 VH) and SEQ ID NO:4 (S01 VL). In some embodiments, the CDRs are as defined using an antibody numbering scheme, such as the Chothia, AbM, Kabat, Contact and IMGT numbering schemes. In some embodiments, the CDRs are as defined using the Kabat numbering scheme. In some embodiments, the VH domain of the target-binding domain comprises a VH-CDR1 comprising the amino acid sequence of SEQ ID NO:32, a VH-CDR2 comprising the amino acid sequence of SEQ ID NO:34, and a VH-CDR3 comprising the amino acid sequence of SEQ ID NO:36, and the VL domain of the target-binding domain comprises a VL-CDR1 comprising the amino acid sequence of SEQ ID NO:39, a VL-CDR2 comprising the amino acid sequence of SEQ ID NO:41, and a VL-CDR3 comprising the amino acid sequence of SEQ ID NO:43.

| Region | SEQ ID | Sequence VH Fragment S01 | Residues | Length |
|---|---|---|---|---|
| HER1 | NO: 31 | HLHLQESGPGLVKPSETLSLTCTVSGGSIN | 1-30 | 30 |
| CDR-H1 | NO: 32 | DTTYYWG | 31-37 | 7 |
| HER2 | NO: 33 | WIRQPPGKGLEWIG | 38-51 | 14 |
| CDR-H2 | NO: 34 | SIYYRGNTHYNSSLRS | 52-67 | 16 |
| HFR3 | NO: 35 | RVTMSVDISKNRFSLKVTSVTAADTAVYYCAR | 68-99 | 32 |
| CDR-H3 | NO: 36 | LDPFDY | 100-105 | 6 |
| HER4 | NO: 37 | WGRGTLVTVSS | 106-116 | 11 |

| Region | SEQ ID | Sequence VL Fragment S01 | Residues | Length |
|---|---|---|---|---|
| LFR1 | NO: 38 | SYVLTQPPSVSVAPGKTARITC | 1-22 | 22 |
| CDR-L1 | NO: 39 | GGNNIGSKSVH | 23-33 | 11 |
| LFR2 | NO: 40 | WYQQKPGQAPVLVIY | 34-48 | 15 |
| CDR-L2 | NO: 41 | YDSDRPS | 49-55 | 7 |
| LFR3 | NO: 42 | GIPERFSGSNSGNTATLTISRVEAGDEADYYC | 56-87 | 32 |
| CDR-L3 | NO: 43 | QVWDSSSDHQV | 88-98 | 11 |
| LFR4 | NO: 44 | FGTGTKVTVL | 99-108 | 11 |

In some embodiments the target binding domain comprises the complementarity-determining regions (CDRs) from VH and VL domains of SEQ ID NO:49 (U01 VH) and SEQ ID NO:50 (U01 VL). In some embodiments, the CDRs are as defined using an antibody numbering scheme, such as the Chothia, AbM, Kabat, Contact and IMGT numbering schemes. In some embodiments, the CDRs are as defined using the Kabat numbering scheme. In some embodiments, the VH domain of the target-binding domain comprises a VH-CDR1 comprising the amino acid sequence of SEQ ID NO:53, a VH-CDR2 comprising the amino acid sequence of SEQ ID NO:55, and a VH-CDR3 comprising the amino acid sequence of SEQ ID NO:57, and the VL domain of the target-binding domain comprises a VL-CDR1 comprising the amino acid sequence of SEQ ID NO:60, a VL-CDR2 comprising the amino acid sequence of SEQ ID NO:62, and a VL-CDR3 comprising the amino acid sequence of SEQ ID NO:64.

| Region | SEQ ID | Sequence VH Fragment U01 | Residues | Length |
|---|---|---|---|---|
| HFR1 | NO: 52 | EVKLIESGGGLVEPGRSERLACTISGFTFA | 1-30 | 30 |
| CDR-H1 | NO: 53 | DYGLS | 31-35 | 5 |
| HFR2 | NO: 54 | WFRQGPGKGLEWVG | 36-49 | 14 |
| CDR-H2 | NO: 55 | FTGPKHLGETTECAPSVED | 50-68 | 19 |
| HFR3 | NO: 56 | RCTISRDDSKSTVYLQMHRIQHEDTAVYFCVG | 69-100 | 32 |
| CDR-H3 | NO: 57 | PWFGDLLM | 101-108 | 8 |
| HFR4 | NO: 58 | WGQGTLVTVSS | 109-119 | 11 |

| Region | SEQ ID | Sequence VH Fragment U01 | Residues | Length |
|---|---|---|---|---|
| LFR1 | NO: 59 | AIQMIQSPSSLSASVGDRVSITC | 1-23 | 23 |
| CDR-L1 | NO: 60 | RATQDISTSIG | 24-34 | 11 |
| LFR2 | NO: 61 | WYHQRPGKAPRLLIY | 35-49 | 15 |
| CDR-L2 | NO: 62 | GASKVQT | 50-56 | 7 |
| LFR3 | NO: 63 | GVPSRFSGNGSGTEFTLTISSLQPEDIGTYYC | 57-88 | 32 |
| CDR-L3 | NO: 64 | LQDDGFPFT | 89-97 | 9 |
| LFR4 | NO: 65 | VGQGTKLDIKRAA | 98-110 | 13 |

In certain embodiments, the target binding domain comprises VH sequences selected from:

(1) SBT01 VH (M)
(SEQ ID NO: 1)
HLHLQESGPGLVKPSETLSLTCTVSGGSINDTTYYWGWIRQPPGKGLEW
IGSIYYRGNTHYNSSLRSRVTMSVDTSKNRFSLKVTSVTAADTAVYYCA
RLDPFDYWGRGTLVTVSS;
(2) SBT01 VH (G)
(SEQ ID NO: 2)
QLQLQESGPGLVKPSETLSLTCTVSGGSISSSSYYWGWIRQPPGKGLEW
IGSIYYSGSTYYNPSLKSRVTISVDTSKNQFSLKLSSVTAADTAVYYCA
RLDPFDYWGRGTLVTVSS;
(3) U01 VH
(SEQ ID NO: 49)
EVKLIESGGGLVEPGRSLRLACTTSGFTFADYGLSWFRQGPGKGLEWVG
FTGPKHLGETTECAPSVEDRCTISRDDSKSTVYLQMHRLQHEDTAVYFC
VGPWFGDLLMWGQGTLVTVSS;

or
a sequence having at least any of 80%, 85%, 90%, 95%, 97%, 98%, 99% or 99.5% sequence identify with the aforementioned sequences, provided target binding domain binds to a citrullinated antigen as described herein.

The target binding region can comprise VL sequences selected from:

(1) SBT01 VL (M)
(SEQ ID NO: 53)
SYVLTQPPSVSLAPGETATITCGGDDIENQNVNWYQQKSGQAPMLLIFF
DTRRPSGIPERFSGSRSEDTANLTITRVEAGDDADYFCQVYDRKTDHQV
FGPGTTVTVL;
(2) SBT01 VL (G)
(SEQ ID NO: 4)
SYVLTQPPSVSVAPGKTARITCGGNNIGSKSVHWYQQKPGQAPVLVIYY
DSDRPSGIPERFSGSNSGNTATLTISRVEAGDEADYYCQVWDSSSDHQV
FGTGTKVTVL;
(3) U01 VL
(SEQ ID NO: 50)
AIQMTQSPSSLSASVGDRVSITCRATQDISTSLGWYHQRPGKAPRLLIY
GASKVQTGVPSRFSGNGSGTEFTLTISSLQPEDIGTYYCLQDDGFPFTV
GQGTKLDIKRAAA;

or
a sequence having at least any of 80%, 85%, 90%, 95%, 97%, 98%, 99% or 99.5% sequence identify with the aforementioned sequences, provided target binding domain binds to a citrullinated antigen as described herein.

In another embodiment, the antigen binding region comprises an scFV comprising one or more VH domains comprising an amino acid sequence of VH domains of SEQ ID NO:1 or SEQ ID NO:2. In some embodiments, the antigen binding region comprises an scFV comprising an amino acid sequence having at least any of 80%, 85%, 90%, 95%, 97%, 98%, 99% or 99.5% of SEQ ID NO:1 or SEQ ID NO:2, provided the scFV domain binds to a citrullinated antigen as described herein.

In some embodiments, the scFV comprises the VL domains of SEQ ID NO:3 or SEQ ID NO:4. In some embodiments, the antigen binding region comprises an scFV comprising an amino acid sequence having at least any of 80%, 85%, 90%, 95%, 97%, 98%, 99% or 99.5% of SEQ ID NO: 3 or SEQ ID NO:4, provided the scFV domain binds to a citrullinated antigen as described herein.

In some embodiments, the scFV domain comprises the VH and VL domains of SEQ ID NO:1 and SEQ ID NO:3, respectively. In some embodiments, the antigen binding region comprises an scFV comprising an amino acid sequence having at least any of 80%, 85%, 90%, 95%, 97%, 98%, 99% or 99.5% of SEQ ID NO: 1 or SEQ ID NO:3, provided the scFV domain binds to a citrullinated antigen as described herein. In some embodiments, a linker selected from SEQ ID NO: 20, SEQ ID NO: 21 or SEQ ID NO: 22 is placed between the VH and VL domains.

In some embodiments, the scFV domain comprises the VH and VL domains of SEQ ID NO:1 and SEQ ID NO:4, respectively. In some embodiments, the antigen binding region comprises an scFV comprising an amino acid sequence having at least any of 80%, 85%, 90%, 95%, 97%, 98%, 99% or 99.5% of SEQ ID NO: 1 and SEQ ID NO:4, provided the scFV domain binds to a citrullinated antigen as described herein. In some embodiments, a linker selected from SEQ ID NO: 20, SEQ ID NO: 21 or SEQ ID NO: 22 is placed between the VH and VL domains.

In some embodiments, the scFV domain comprises the VH and VL domains of SEQ ID NO: 2 and SEQ ID NO:3, respectively. In some embodiments, the antigen binding region comprises an scFV comprising an amino acid sequence having at least any of 80%, 85%, 90%, 95%, 97%, 98%, 99% or 99.5% of SEQ ID NO: 2 and SEQ ID NO: 3, provided the scFV domain binds to a citrullinated antigen as described herein. In some embodiments, a linker selected from SEQ ID NO: 20, SEQ ID NO: 21 or SEQ ID NO: 22 is placed between the VH and VL domains.

In some embodiments, the scFV domain comprises the VH and VL domains of SEQ ID NO:2 and SEQ ID NO:4, respectively. In some embodiments, the antigen binding region comprises an scFV comprising an amino acid sequence having at least any of 80%, 85%, 90%, 95%, 97%, 98%, 99% or 99.5% of SEQ ID NO: 2 and SEQ ID NO: 4, provided the scFV domain binds to a citrullinated antigen as described herein. In some embodiments, a linker selected from SEQ ID NO: 20, SEQ ID NO: 21 or SEQ ID NO: 22 is placed between the VH and VL domains.

In some embodiments, the scFV domain comprises the VH and VL domains of SEQ ID NO:49 and SEQ ID NO:50, respectively. In some embodiments, the antigen binding region comprises an scFV comprising an amino acid sequence having at least any of 80%, 85%, 90%, 95%, 97%, 98%, 99% or 99.5% of SEQ ID NO: 49 and SEQ ID NO: 50, provided the scFV domain binds to a citrullinated antigen as described herein. In some embodiments, a linker selected from SEQ ID NO: 20, SEQ ID NO: 21 or SEQ ID NO: 22 is placed between the VH and VL domains.

In another embodiment, the antigen binding region comprises an scFV comprising an amino acid sequence selected from:

SBT01G-VHVL-GGGSx3 Linker-pSB_0149
(SEQ ID NO: 5)
HLHLQESGPGLVKPSETLSLTCTVSGGSINDTTYYWGWIRQPPGKGLEW
IGSIYYRGNTHYNSSLRSRVTMSVDTSKNRFSLKVTSVTAADTAVYYCA
RLDPFDYWGRGTLVTVSSGGGGSGGGGSGGGGSSYVLTQPPSVSVAPGK
TARITCGGNNIGSKSVHWYQQKPGQAPVLVIYYDSDRPSGIPERFSGSN
SGNTATLTISRVEAGDEADYYCQVWDSSSDHQVFGTGTKVTVLR
(GGGSx3 linker in bold);
SBT01G-VHVL-Whitlow 218 Linker-pSB_0158
(SEQ ID NO: 6)
HLHLQESGPGLVKPSETLSLTCTVSGGSINDTTYYWGWIRQPPGKGLEW
IGSIYYRGNTHYNSSLRSRVTMSVDTSKNRFSLKVTSVTAADTAVYYCA
RLDPFDYWGRGTLVTVSSGSTSGSGKPGSGEGSTKGSYVLTQPPSVSVA
PGKTARITCGGNNIGSKSVHWYQQKPGQAPVLVIYYDSDRPSGIPERFS
GSNSGNTATLTISRVEAGDEADYYCQVWDSSSDHQVFGTGTKVTVLR
(Whitlow 218 Linker in bold);
SBT01G-VHVL-AB pur Linker-pSB_0159
(SEQ ID NO: 7)
HLHLQESGPGLVKPSETLSLTCTVSGGSINDTTYYWGWIRQPPGKGLEW
IGSIYYRGNTHYNSSLRSRVTMSVDTSKNRFSLKVTSVTAADTAVYYCA
RLDPFDYWGRGTLVTVSSASSGGSTSGSGKPGSGEGSSGSARSYVLTQP
PSVSVAPGKTARITCGGNNIGSKSVHWYQQKPGQAPVLVIYYDSDRPSG
IPERFSGSNSGNTATLTISRVEAGDEADYYCQVWDSSSDHQVFGTGTKV
TVLR
(AB pur Linker in bold);
U01-VHVL-Ab pur Linker
(SEQ ID NO: 51)
EVKLIESGGGLVEPGRSLRLACTTSGFTFADYGLSWFRQGPGKGLEWVG
FTGPKHLGETTECAPSVEDRCTISRDDSKSTVYLQMHRLQHEDTAVYFC
VGPWFGDLLMWGQGTLVTVSSASSGGSTSGSGKPGSGEGSSGSARAIQM
TQSPSSLSASVGDRVSITCRATQDISTSLGWYHQRPGKAPRLLIYGASK
VQTGVPSRFSGNGSGTEFTLTISSLQPEDIGTYYCLQDDGFPFTVGQGT
KLDIKRAAA
(AB pur Linker in bold);

or
a sequence having at least any of 80%, 85%, 90%, 95%, 97%, 98%, 99% or 99.5% sequence identify with the aforementioned sequences, provided target binding domain binds to a citrullinated antigen as described herein.

For example, the linker can comprise the sequence:

GGGSx3 Linker
(SEQ ID NO: 20)
GGGGSGGGGSGGGGS;
or
Whitlow 218 Linker
(SEQ ID NO: 21)
GSTSGSGKPGSGEGSTKG;
or
AB pur Linker
(SEQ ID NO: 22)
ASSGGSTSGSGKPGSGEGSSGSAR.

Optionally, any of the foregoing sequences can include the CDR sets from the VH and VL domains described above.

### C. Hinge Region

In some embodiments, the hinge region of the disclosed CARs can be selected from the CD8, CD4, or CD28 extracellular domain, the Fc region of an IgG1 antibody, or the extracellular domain of any of the TLR receptors as is known to one of skill in the art and can be found in the GenBank database.

For example, the hinge region can comprise the sequence:

(CD28)
(SEQ ID NO: 30)
IEVMYPPPYLDNEKSNGTIIHVKGKHLCPSPLFPGPSKP;
or
(CD8)
(SEQ ID NO: 15)
TTTPAPRPPTPAPTIASQPLSLRPEACRPAAGGAVHTRGLDFACDIYI
WAPLAGTCGVLLLSLVITLYC;

or
a sequence having at least any of 80%, 85%, 90%, 95%, 97%, 98%, 99% or 99.5% sequence identify with the aforementioned hinge region sequences.

### D. Transmembrane Domain

The transmembrane domain can comprise a transmembrane domain of an immunoglobulin family receptor, such as CD8. The intracellular domain can be selected from any membrane-spanning molecule on a T cell. For example, the transmembrane (TM) domain of the disclosed CAR can comprise the TM domain of CD2, CD3, CD16, CD32, CD64, CD28, CD247, 4-1 BBL, CD4, or CD8.

For example, the transmembrane domain can comprise the sequence:

(SEQ ID NO: 16)
FWVLVVVGGVLACYSLLVTVAFIIFWV;
or
(SEQ ID NO: 17)
IYIWAPLAGTCGVLLLSLVITLYC;

or
a sequence having at least any of 80%, 85%, 90%, 95%, 97%, 98%, 99% or 99.5% sequence identify with the aforementioned hinge region sequences.

### E. Signal Transduction Domain

### 1. CD3ζ Signal Transduction Domain

In some embodiments, the signal transduction domain comprises a CD3ζ signaling domain. The CD3ζ signaling domain of the disclosed CAR molecule can comprise a CD3ζ amino acid sequence, e.g., a signal transduction domain of CD3 zeta.

For example, the CD3 signal transduction domain can comprise the sequence:

(SEQ ID NO: 19)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR;

or
a sequence having at least any of 80%, 85%, 90%, 95%, 97%, 98%, 99% or 99.5% sequence identify with the aforementioned CD3 zeta signal transduction domain sequence.

For example, the CD3 zeta signal transduction domain can include amino acids 21-163, 31-142, 68-89 and/or 138-158 of the sequence shown in SEQ ID NO:19, or functional variants thereof (e.g., with 1, 2, 3, 4, 5, 6, 7, 8, 9, 10, or 10-20 amino acid substitutions, deletions, or additions).

### 2. Fc Receptor Signal Transduction Domain

In some embodiments, the signal transduction domain comprises an Fc signaling domain. The Fc signaling domain can be any one of the Fc-alpha, Fc-gamma, Fc-epsilon, Fc-mu, and Fc-delta receptors. For example, the Fc receptor signaling domain can comprise amino acids involved in interaction with Src (e.g., Fgr, Fyn, Hck, Lyn, Yes, and Src) and ZAP-70 family kinases, e.g., one or more ITAM domains (see, e.g., Sanchez-Mejorada et al. (1998) J. Leukocyte Biol. 63:531; Garcia-Garcia et al. (2002) J. Leukocyte Biol. 72:1092). In some embodiments, the Fc receptor signal transduction domain includes at least one ITAM domain, e.g., from any one of the Fc-alpha, Fc-gamma, Fc-epsilon, Fc-mu, and Fc-delta receptors, or substantially identical thereto.

Sequences also can be found as follows:

| Molecule | GenBank No. |
|---|---|
| CD3ζ | NP_000725.1 |
| Fc gamma receptor family (CD16) | NP_000560.5 |
| | NP_001231682.1 |
| Fc gamma receptor family (CD32) | AAH20823.1 |
| | AAH19931.1 |
| | AAI48274.1 |
| | AAI37398.1 |
| Fc gamma receptor family (CD64) | AAI60240.1 |
| | AAH32634.1 |
| | AAI56865.1 |

### F. Co-Stimulatory Domain

The CARs of this disclosure can include one or more co-stimulatory domains in addition to a signal transduction domain of CD3ζ or an Fc receptor. Co-stimulatory domains can be derived from, for example, CD28, 4-1 BB, CD2, CD27, CD30, OX40, CD40, PD-1, PD-L1, PD-L2, ICOS, LFA-1, CD7, LIGHT, NKG2C, B7-H3, CD83L, B7-1 (CD80), B7-2 (CD86), B7-H3, B7-H4 and others. The CAR constructs can contain two or more co-stimulatory signaling domains (e.g., CD28 and 4-1 BB).

The co-stimulatory domain or domains can be positioned between the signal transduction domain and the transmembrane region.

In certain embodiments, a CD28 co-stimulatory domain can comprise the sequence:

(SEQ ID NO: 29)
RSKRSRLLHSDYMNMTPRRPGPTRKHYQPYAPPRDFAAYRS;

or
a sequence having at least any of 80%, 85%, 90%, 95%, 97%, 98%, 99% or 99.5% sequence identify with the aforementioned sequence.

In certain embodiments, a 41 BB co-stimulator domain can comprise the sequence:

(SEQ ID NO: 28)
KRGRKKLLYIFKQPFMRPVQTTQEEDGCSCRFPEEEEGGCEL;

or
a sequence having at least any of 80%, 85%, 90%, 95%, 97%, 98%, 99% or 99.5% sequence identify with the aforementioned sequence.

### III. NUCLEIC ACIDS

### A. Nucleic Acids Encoding CARs

Disclosed herein are nucleic acid molecules (polynucleotides) comprising a nucleotide sequence that encodes a CAR of this disclosure. The nucleic acid of the disclosed CAR can be in the form of DNA or in the form of RNA. DNA includes cDNA, genomic DNA, and synthetic DNA; and can be double-stranded or single-stranded, and if single stranded can be the coding strand or non-coding (anti-sense) strand. RNA includes mRNA, siRNA, sRNA, ssRNA and so on.

For example, nucleic acids can comprise nucleotide sequences that encode the polypeptides of any of SEQ ID NO:1, SEQ ID NO:2, SEQ ID NO:3, SEQ ID NO:4. In certain embodiments, the nucleotide sequences encoding VH domains comprise:

(1) SBT01 VH (M)
(SEQ ID NO: 8)
CACCTGCACTTGCAGGAGTCGGGCCCAGGACTTGTGAAGCCTTCGGAGA
CCCTGTCCCTCACCTGCACTGTCTCTGGTGGCTCCATCAACGATACCAC
TTACTACTGGGGCTGGATTCGCCAGCCCCCCGGGAAGGGACTGGAGTGG
ATTGGGAGTATCTATTACCGGGGGAACACCCACTACAATTCGTCCCTGA
GGAGTCGCGTCACCATGTCTGTCGACACTTCCAAGAACCGATTCTCCCT
GAAGGTCACTTCTGTGACTGCCGCAGACACGGCTGTCTATTACTGTGCG
AGACTCGACCCATTTGACTACTGGGGCCGTGGCACCCTGGTCACTGTCT
CGAGC,
(2) SBT01 VH (G)
(SEQ ID NO: 9)
CAGCTGCAGCTGCAGGAGTCGGGCCCAGGACTGGTGAAGCCTTCGGAGA
CCCTGTCCCTCACCTGCACTGTCTCTGGTGGCTCCATCAGCAGTAGTAG
TTACTACTGGGGCTGGATCCGCCAGCCCCCAGGGAAGGGGCTGGAGTGG
ATTGGGAGTATCTATTATAGTGGGAGCACCTACTACAACCCGTCCCTCA
AGAGTCGAGTCACCATATCCGTAGACACGTCCAAGAACCAGTTCTCCCT
GAAGCTGAGCTCTGTGACCGCCGCAGACACGGCTGTGTATTACTGTGCG
AGACTCGACCCATTTGACTACTGGGGCCGTGGCACCCTGGTCACTGTCT
CGAGC;

or
a sequence having at least any of 80%, 85%, 90%, 95%, 97%, 98%, 99% or 99.5% sequence identify with the aforementioned sequences.

In certain embodiments, the nucleotide sequences encoding VL domains comprise:

(1) SBT01 VL (M)
(SEQ ID NO: 10)
TCCTATGTCCTGACTCAGCCACCCTCAGTGTCGCTGGCCCCGGGAGAGA
CGGCCACAATTACTTGTGGTGGAGACGACATTGAAAATCAAAATGTCAA
CTGGTATCAGCAGAAGTCAGGTCAGGCCCCTATGCTGCTCATCTTCTTT
GATACCAGACGGCCCTCAGGGATCCCGGAGCGATTCTCTGGCTCCAGGT
CTGAGGACACGGCCAACCTGACCATCACCAGGGTCGAGGCCGGGGATGA
CGCCGACTATTTCTGTCAGGTGTATGATAGGAAGACTGATCACCAAGTC
TTCGGACCTGGGACCACGGTCACCGTCCTA;
(2) SBT01 VL (G)
(SEQ ID NO: 11)
TCCTATGTGCTGACTCAGCCACCCTCAGTGTCAGTGGCCCCAGGAAAGA
CGGCCAGGATTACCTGTGGGGGAAACAACATTGGAAGTAAAAGTGTGCA
CTGGTACCAGCAGAAGCCAGGCCAGGCCCCTGTGCTGGTCATCTATTAT
GATAGCGACCGGCCCTCAGGGATCCCTGAGCGATTCTCTGGCTCCAACT
CTGGGAACACGGCCACCCTGACCATCAGCAGGGTCGAAGCCGGGGATGA
GGCCGACTATTACTGTCAGGTGTGGGACAGTAGTAGTGATCACCAAGTC
TTCGGAACTGGGACCAAGGTCACCGTCCTA;

or
a sequence having at least any of 80%, 85%, 90%, 95%, 97%, 98%, 99% or 99.5% sequence identify with the aforementioned sequences.

In another embodiment, the nucleic acid molecule encoding VH domains comprise SEQ ID NO: 8 or SEQ ID NO:9. In some embodiments, the nucleic acid molecule encoding VH domains comprise a nucleic sequence having at least any of 80%, 85%, 90%, 95%, 97%, 98%, 99% or 99.5% of SEQ ID NO:8 or SEQ ID NO:9, provided the scFV domain binds to a citrullinated antigen as described herein.

In some embodiments, the nucleic acid molecule encoding VL domains comprise SEQ ID NO: 10 or SEQ ID NO: 11. In some embodiments, the nucleic acid molecule encoding VL domains comprise a nucleic sequence having at least any 80%, 85%, 90%, 95%, 97%, 98%, 99% or 99.5% of SEQ ID NO: 10 or SEQ ID NO: 11, provided the scFV domain binds to a citrullinated antigen as described herein.

In some embodiments, the nucleic acid molecule encoding an scFV domain comprise SEQ ID NO:8 and SEQ ID NO:10. In some embodiments, the nucleic acid molecule encoding the scFV comprises an amino acid sequence having at least any of 80%, 85%, 90%, 95%, 97%, 98%, 99% or 99.5% of SEQ ID NO: 8 or SEQ ID NO:10, provided the scFV domain binds to a citrullinated antigen as described herein.

In some embodiments, the nucleic acid molecule encoding an scFV domain comprise SEQ ID NO: 8 and SEQ ID NO: 11. In some embodiments, the nucleic acid molecule encoding the scFV comprises an amino acid sequence having at least any of 80%, 85%, 90%, 95%, 97%, 98%, 99% or 99.5% of SEQ ID NO: 8 or SEQ ID NO:11, provided the scFV domain binds to a citrullinated antigen as described herein.

In some embodiments, the nucleic acid molecule encoding an scFV domain comprise SEQ ID NO: 9 and SEQ ID NO:10. In some embodiments, the nucleic acid molecule encoding the scFV comprises an amino acid sequence having at least any of 80%, 85%, 90%, 95%, 97%, 98%, 99% or 99.5% of SEQ ID NO: 9 or SEQ ID NO: 10, provided the scFV domain binds to a citrullinated antigen as described herein.

In some embodiments, the nucleic acid molecule encoding an scFV domain comprise SEQ ID NO: 9 and SEQ ID NO:11. In some embodiments, the nucleic acid molecule encoding the scFV comprises an amino acid sequence having at least any of 80%, 85%, 90%, 95%, 97%, 98%, 99% or 99.5% of SEQ ID NO: 9 or SEQ ID NO: 11, provided the scFV domain binds to a citrullinated antigen as described herein.

In another embodiment, the nucleic acid molecule encodes an scFv molecule and has a nucleotide sequence:

SBT01G-VHVL-GGGSx3 Linker-pSB_0149
(SEQ ID NO: 12)
CACCTGCACTTGCAGGAGTCGGGCCCAGGACTTGTGAAGCCTTCGGAGA
CCCTGTCCCTCACCTGCACTGTCTCTGGTGGCTCCATCAACGATACCAC
TTACTACTGGGGCTGGATTCGCCAGCCCCCCGGGAAGGGACTGGAGTGG
ATTGGGAGTATCTATTACCGGGGGAACACCCACTACAATTCGTCCCTGA
GGAGTCGCGTCACCATGTCTGTCGACACTTCCAAGAACCGATTCTCCCT
GAAGGTCACTTCTGTGACTGCCGCAGACACGGCTGTCTATTACTGTGCG
AGACTCGACCCATTTGACTACTGGGGCCGTGGCACCCTGGTCACTGTCT
CGAGCGGTGGCGGTGGCTCGGGCGGTGGTGGGTCGGGTGGCGGCGGATC
TTCCTATGTGCTGACTCAGCCACCCTCAGTGTCAGTGGCCCCAGGAAAG
ACGGCCAGGATTACCTGTGGGGGAAACAACATTGGAAGTAAAAGTGTGC
ACTGGTACCAGCAGAAGCCAGGCCAGGCCCCTGTGCTGGTCATCTATTA
TGATAGCGACCGGCCCTCAGGGATCCCTGAGCGATTCTCTGGCTCCAAC
TCTGGGAACACGGCCACCCTGACCATCAGCAGGGTCGAAGCCGGGGATG
AGGCCGACTATTACTGTCAGGTGTGGGACAGTAGTAGTGATCACCAAGT
CTTCGGAACTGGGACCAAGGTCACCGTCCTACGCG
(GGGSx3 linker underlined);

or
a sequence having at least any of 80%, 85%, 90%, 95%, 97%, 98%, 99% or 99.5% sequence identify with the aforementioned sequence.

In another embodiment, the nucleic acid molecule encodes an scFv molecule and has a nucleotide sequence:

SBT01G-VHVL-Whitlow 218 Linker-pSB_0158
(SEQ ID NO: 13)
CACCTGCACTTGCAGGAGTCGGGCCCAGGACTTGTGAAGCCTTCGGAG
ACCCTGTCCCTCACCTGCACTGTCTCTGGTGGCTCCATCAACGATACC
ACTTACTACTGGGGCTGGATTCGCCAGCCCCCCGGGAAGGGACTGGAG
TGGATTGGGAGTATCTATTACCGGGGGAACACCCACTACAATTCGTCC
CTGAGGAGTCGCGTCACCATGTCTGTCGACACTTCCAAGAACCGATTC
TCCCTGAAGGTCACTTCTGTGACTGCCGCAGACACGGCTGTCTATTAC
TGTGCGAGACTCGACCCATTTGACTACTGGGGCCGTGGCACCCTGGTC
ACTGTCTCGAGCGGAAGCACGAGTGGTTCAGGCAAACCGGGTTCCGGT
GAAGGTTCAACAAAAGGTTCCTATGTGCTGACTCAGCCACCCTCAGTG
TCAGTGGCCCCAGGAAAGACGGCCAGGATTACCTGTGGGGGAAACAAC
ATTGGAAGTAAAAGTGTGCACTGGTACCAGCAGAAGCCAGGCCAGGCC
CCTGTGCTGGTCATCTATTATGATAGCGACCGGCCCTCAGGGATCCCT
GAGCGATTCTCTGGCTCCAACTCTGGGAACACGGCCACCCTGACCATC
AGCAGGGTCGAAGCCGGGGATGAGGCCGACTATTACTGTCAGGTGTGG
GACAGTAGTAGTGATCACCAAGTCTTCGGAACTGGGACCAAGGTCACC
GTCCTACGC
(Whitlow 218 Linker underlined);

or
a sequence having at least any of 80%, 85%, 90%, 95%, 97%, 98%, 99% or 99.5% sequence identify with the aforementioned sequence.

In another embodiment, the nucleic acid molecule encodes an scFv molecule and has a nucleotide sequence:

SBT01G-VHVL-AB pur Linker-pSB_0159
(SEQ ID NO: 14)
CACCTGCACTTGCAGGAGTCGGGCCCAGGACTTGTGAAGCCTTCGGAGA
CCCTGTCCCTCACCTGCACTGTCTCTGGTGGCTCCATCAACGATACCAC
TTACTACTGGGGCTGGATTCGCCAGCCCCCCGGGAAGGGACTGGAGTGG
ATTGGGAGTATCTATTACCGGGGGAACACCCACTACAATTCGTCCCTGA
GGAGTCGCGTCACCATGTCTGTCGACACTTCCAAGAACCGATTCTCCCT
GAAGGTCACTTCTGTGACTGCCGCAGACACGGCTGTCTATTACTGTGCG
AGACTCGACCCATTTGACTACTGGGGCCGTGGCACCCTGGTCACTGTCT
CGAGCGCCTCTAGCGGGGGGAGCACATCAGGAAGCGGCAAGCCCGGTAG
CGGCGAAGGCTCCTCTGGCAGCGCCCGCTCCTATGTGCTGACTCAGCCA
CCCTCAGTGTCAGTGGCCCCAGGAAAGACGGCCAGGATTACCTGTGGGG
GAAACAACATTGGAAGTAAAAGTGTGCACTGGTACCAGCAGAAGCCAGG
CCAGGCCCCTGTGCTGGTCATCTATTATGATAGCGACCGGCCCTCAGGG
ATCCCTGAGCGATTCTCTGGCTCCAACTCTGGGAACACGGCCACCCTGA
CCATCAGCAGGGTCGAAGCCGGGGATGAGGCCGACTATTACTGTCAGGT
GTGGGACAGTAGTAGTGATCACCAAGTCTTCGGAACTGGGACCAAGGTC
ACCGTCCTACGC
(AB pur Linker underlined);

or
a sequence having at least any of 80%, 85%, 90%, 95%, 97%, 98%, 99% or 99.5% sequence identify with the aforementioned sequences.

Optionally, these can include sequences encoding the CDR sets from the VH and VL domains described herein.

The polynucleotide variants can contain alterations in the coding regions, non-coding regions, or both. In some embodiments, the polynucleotide variants contain alterations that produce silent substitutions, additions, or deletions, but do not alter the properties or activities of the encoded CAR polypeptide. In some embodiments, the polynucleotide variants contain alterations that do not produce any changes in the amino acid sequence. In some embodiments, polynucleotide variants contain “silent” substitutions due to the degeneracy of the genetic code. Polynucleotide variants can be produced for a variety of reasons, for example, to optimize codon expression for a particular host.

In some embodiments, the polynucleotides as described herein are isolated.

Polynucleotides encoding CARs be isolated molecules, or can be included within a vector, such as a plasmid, a cosmid, an artificial chromosome or a virus. Such vectors can be used to transfect target cells.

### B. Expression Constructs and Vectors

Polynucleotides encoding CARs of this disclosure can include regulatory elements operatively linked with a nucleotide sequence encoding the CAR. For example, a polynucleotide can include one or more transcription regulatory elements, such as promoters or enhancers, which, when the polynucleotide is present in a cell, cause the sequence encoding the CAR to be expressed within the cell.

Nucleic acids disclosed herein can be incorporated into vectors capable of transfecting cells. Such vectors include, without limitation, viral vectors plasmids and microvesicles, e.g., liposomes. Exemplary viral vectors adenoviral vectors Ad, AAV, lentivirus, and vesicular stomatitis virus (VSV) and retroviruses. Lentiviruses are a genus of the Retroviridae family and include HIV, SIV, and FIV. Lentiviruses can deliver a large quantities of genetic material into the DNA of the host cell. They are able to infect non-dividing cells.

### IV. CELLS

In some embodiments, the recombinant (host) cell having the nucleic acid molecule encoding the disclosed CAR wherein the nucleic acid molecule can further comprise an expression control sequence operatively linked with the nucleotide sequence encoding the CAR. The assembled CAR (by synthesis, site-directed mutagenesis or another method, as is known to one of skill in the art), the nucleic acid molecule encoding the disclosed CAR can be inserted into an expression vector and operatively linked to an expression control sequence appropriate for expression of the disclosed CAR in a desired host. Correct assembly can be confirmed by nucleotide sequencing, restriction mapping, and/or expression of the CAR polypeptide in a suitable host. As is well known in the art, in order to obtain high expression levels of a transfected gene in a host, the gene must be operatively linked to transcriptional and translational expression control sequences that are functional in the chosen expression host.

This disclosure also provides cells (e.g., recombinant cells) comprising nucleic acid molecules encoding CARs and/or expressing CARs.

The nucleic acid molecule encoding the disclosed CAR can be delivered to a host cell, including but not limited to a T cell, B cell, myeloid progenitor, macrophage, and so on, by a plasmid or a viral vector as is known to one of skill in the art. The resulting recombinant (host) cell can include but is not limited to a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, a T memory stem cell as well as cells expressing MHC class I or class II as is known to one of skill in the art. In some embodiments, the recombinant (host) cell having the nucleic acid molecule encoding the disclosed CAR can be a myeloid progenitor cell selected from the group consisting of a common myeloid progenitor, a granulocyte macrophage progenitor, a megakaryocyte erythrocyte progenitor, a granulocyte progenitor and a monocyte progenitor as is known to one of skill in the art. In some embodiments, the myeloid cell is an autologous or allogeneic cell.

In some embodiments, the cells expressing the CARs of this disclosure are Treg cells. “Regulatory T cells,” or “T_{reg} cells,” are cells belonging to a specialized subpopulation of T cells that act to suppress immune response, thereby maintaining homeostasis and self-tolerance. T_{reg}s are able to inhibit T cell proliferation and cytokine production and play a critical role in preventing autoimmunity. T_{reg}s are characterized by expression of FoxP3. Surface markers for T_{reg}s include CD4, CD25high (high molecular density) and CD127low (low molecular density). Mouse and human Tregs express GITR/AITR, and CTLA-4. Human CD4+FoxP3+ Treg cells can be divided into three sub-populations: (1) CD45RA+CD25+FoxP310w resting Treg cells, (2) CD45RO+CD25highFoxP3high activated Treg cells, and (3) proinflammatory cytokine-producing CD45RO+CD25+FoxP3low nonsuppressive effector T cells (Teffs).

The cells to be transformed with the nucleic acids disclosed herein can be cells taken from a subject into whom the recombinant cells are to be administered. In this way, issues of an allogeneic immune response can be mitigated.

Cells can be expanded ex vivo before administration to a subject.

Treg cells into which nucleic acids expressing the CARs of this disclosure have been incorporated can express these CARs and be used in the methods described herein to treat hidradenitis suppurativa.

Also, the proteins produced by a transformed/recombinant host can be purified according to any suitable method. Such methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence and glutathione-S-transferase can be attached to the protein to allow easy purification by passage over an appropriate affinity column. In some embodiments, proteins can also be physically characterized using such techniques as proteolysis, high performance liquid chromatography (HPLC), nuclear magnetic resonance and x-ray crystallography.

### V. COMPOSITIONS

Also disclosed are pharmaceutical compositions comprising a recombinant cell having a nucleic acid molecule encoding the disclosed CAR polypeptide and/or expressing the disclosed CAR polypeptide, and a pharmaceutically acceptable carrier, as well as methods of use in the treatment hidradenitis suppurativa.

As used herein, the term “pharmaceutical composition” refers to a composition comprising a pharmaceutical compound (e.g., a drug or a recombinant Treg cell as described herein) and a pharmaceutically acceptable carrier.

As used herein, the term “pharmaceutically acceptable” refers to a carrier that is compatible with the other ingredients of a pharmaceutical composition and can be safely administered to a subject. The term is used synonymously with “physiologically acceptable” and “pharmacologically acceptable”. Pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

Pharmaceutically acceptable carriers will generally be sterile, at least for human use. A pharmaceutical composition will generally comprise agents for buffering and preservation in storage, and can include buffers and carriers for appropriate delivery, depending on the route of administration. Examples of pharmaceutically acceptable carriers include, without limitation, normal (0.9%) saline, phosphate-buffered saline (PBS) Hank's balanced salt solution (HBSS) and multiple electrolyte solutions such as PlasmaLyte ATM (Baxter).

Pharmaceutical compositions can be formulated for any route of administration, including mucosal (e.g., nasal, sublingual, vaginal, buccal, or rectal), parenteral (e.g., subcutaneous, intravenous, intramuscular, or intraarterial injection, either bolus or infusion), oral, or transdermal.

Injectable (e.g., intravenous) compositions can comprise a solution of the composition suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) will be used. The compositions can contain pharmaceutically acceptable auxiliary substances to approximate physiological conditions, such as pH adjusting and buffering agents, tonicity adjusting agents, wetting agents, e.g., sodium acetate, sodium lactate, sodium chloride, potassium chloride, calcium chloride, sorbitan monolaurate, triethanolamine oleate, etc. In some embodiments, the composition can be formulated in a kit for intravenous administration.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. The formulations of compositions can be presented in unit-dose or multi-dose sealed containers, such as ampoules and vials.

Cells can be cryopreserved. Cryopreservation can include formulating cells with a cryopreservation agent, such as DMSO. Commercially available media include, for example, CryoStor® and pZerve®, available from Millipore Sigma.

Compositions can be formulated as dosage forms for administration. The term “dosage form” refers to the particular format of the pharmaceutical, and depends on the route of administration. Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The terms “dose” and “dosage” are used interchangeably herein. A dose refers to the amount of active ingredient given to an individual at each administration. The dose will vary depending on a number of factors, including frequency of administration; size and tolerance of the individual; severity of the condition; risk of side effects; the route of administration; and the imaging modality of the detectable label (if present). One of skill in the art will recognize that the dose can be modified depending on the above factors or based on therapeutic progress.

The pharmaceutical preparation can be packaged or prepared in unit dosage form. In such form, the preparation is subdivided into unit doses containing appropriate quantities of the active component, e.g., according to the dose of the therapeutic agent or concentration of the composition. The unit dosage form can be a packaged preparation, the package containing discrete quantities of preparation. The composition can, if desired, also contain other compatible therapeutic agents.

In some embodiments, the compositions of the invention comprise a recombinant cell having a nucleic acid molecule encoding a CAR polypeptide comprising the complementarity-determining regions (CDRs) from the VH domains of SEQ ID NO:1 or SEQ ID NO:2. In some embodiments, the compositions of the invention comprise a recombinant cell having a nucleic acid molecule encoding a CAR polypeptide comprising the CDRs from the VL domains of SEQ ID NO:3 or SEQ ID NO:4. In some embodiments, the compositions of the invention comprise a recombinant cell having a nucleic acid molecule encoding a CAR polypeptide comprising the CDRs from VH and VL domains of SEQ ID NO:1 and SEQ ID NO:3. In some embodiments, the compositions of the invention comprise a recombinant cell having a nucleic acid molecule encoding a CAR polypeptide comprising the CDRs from VH and VL domains of SEQ ID NO:1 and SEQ ID NO:4. In some embodiments, the compositions of the invention comprise a recombinant cell having a nucleic acid molecule encoding a CAR polypeptide comprising the CDRs from VH and VL domains of SEQ ID NO:2 and SEQ ID NO:3. In some embodiments, the compositions of the invention comprise a recombinant cell having a nucleic acid molecule encoding a CAR polypeptide comprising the CDRs from VH and VL domains of SEQ ID NO:2 and SEQ ID NO:4. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

In some embodiments, the compositions of the invention comprise a recombinant cell having a nucleic acid molecule encoding a CAR polypeptide comprising the complementarity-determining regions (CDRs) from the VH domains of SEQ ID NO:49. In some embodiments, the compositions of the invention comprise a recombinant cell having a nucleic acid molecule encoding a CAR polypeptide comprising the CDRs from the VL domains of SEQ ID NO:50. In some embodiments, the compositions of the invention comprise a recombinant cell having a nucleic acid molecule encoding a CAR polypeptide comprising the CDRs from VH and VL domains of SEQ ID NO:49 and SEQ ID NO:50. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

In some embodiments, the compositions of the invention comprise a recombinant cell having a nucleic acid molecule encoding a CAR polypeptide comprising the CDRs from VH and VL domains of SEQ ID NO:1 (SBT01 VH (M)) and SEQ ID NO:4 (SBT01 VL (G)). In some embodiments, the VH domain of the CAR polypeptide comprises a VH-CDR1 comprising the amino acid sequence of SEQ ID NO:32, a VH-CDR2 comprising the amino acid sequence of SEQ ID NO:34, and a VH-CDR3 comprising the amino acid sequence of SEQ ID NO:36, and the VL domain of CAR polypeptide comprises a VL-CDR1 comprising the amino acid sequence of SEQ ID NO:39, a VL-CDR2 comprising the amino acid sequence of SEQ ID NO:41, and a VL-CDR3 comprising the amino acid sequence of SEQ ID NO:43. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

In another embodiment, the compositions of the invention comprise a recombinant cell having a nucleic acid molecule encoding a CAR polypeptide comprising an scFV comprising one or more VH domains comprising the amino acid sequence of VH domains of SEQ ID NO:1 or SEQ ID NO:2. In some embodiments, the nucleic acid molecule encoding a CAR polypeptide comprises an scFV comprising an amino acid sequence having at least any of 80%, 85%, 90%, 95%, 97%, 98%, 99% or 99.5% of SEQ ID NO:1 or SEQ ID NO:2, provided the scFV domain binds to a citrullinated antigen as described herein. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

In some embodiments, the compositions of the invention comprise a recombinant cell having a nucleic acid molecule encoding a CAR polypeptide comprising an scFV comprising the VL domains of SEQ ID NO:3 or SEQ ID NO:4. In some embodiments, the nucleic acid molecule encoding a CAR polypeptide comprises an scFV comprising an amino acid sequence having at least any of 80%, 85%, 90%, 95%, 97%, 98%, 99% or 99.5% of SEQ ID NO: 3 or SEQ ID NO:4, provided the scFV domain binds to a citrullinated antigen as described herein. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

In some embodiments, the compositions of the invention comprise a recombinant cell having a nucleic acid molecule encoding a CAR polypeptide comprising an scFV comprising the VH and VL domains of SEQ ID NO:1 and SEQ ID NO:3. In some embodiments, the nucleic acid molecule encoding a CAR polypeptide comprises an scFV comprising an amino acid sequence having at least any of 80%, 85%, 90%, 95%, 97%, 98%, 99% or 99.5% of SEQ ID NO: 1 or SEQ ID NO:3, provided the scFV domain binds to a citrullinated antigen as described herein. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

In some embodiments, the compositions of the invention comprise a recombinant cell having a nucleic acid molecule encoding a CAR polypeptide comprising an scFV comprising the VH and VL domains of SEQ ID NO:1 and SEQ ID NO:4. In some embodiments, the nucleic acid molecule encoding a CAR polypeptide comprises an scFV comprising an amino acid sequence having at least any of 80%, 85%, 90%, 95%, 97%, 98%, 99% or 99.5% of SEQ ID NO: 1 and SEQ ID NO:4, provided the scFV domain binds to a citrullinated antigen as described herein. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

In some embodiments, the compositions of the invention comprise a recombinant cell having a nucleic acid molecule encoding a CAR polypeptide comprising an scFV comprising the VH and VL domains of SEQ ID NO: 2 and SEQ ID NO:3. In some embodiments, the nucleic acid molecule encoding a CAR polypeptide comprises an scFV comprising an amino acid sequence having at least any of 80%, 85%, 90%, 95%, 97%, 98%, 99% or 99.5% of SEQ ID NO: 2 and SEQ ID NO: 3, provided the scFV domain binds to a citrullinated antigen as described herein. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

In some embodiments, the compositions of the invention comprise a recombinant cell having a nucleic acid molecule encoding a CAR polypeptide comprising an scFV comprising the VH and VL domains of SEQ ID NO:2 and SEQ ID NO:4. In some embodiments, the nucleic acid molecule encoding a CAR polypeptide comprises an scFV comprising an amino acid sequence having at least any of 80%, 85%, 90%, 95%, 97%, 98%, 99% or 99.5% of SEQ ID NO: 2 and SEQ ID NO: 4, provided the scFV domain binds to a citrullinated antigen as described herein. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

In some embodiments, the compositions of the invention comprise a recombinant cell having a nucleic acid molecule encoding a CAR polypeptide comprising an scFV comprising the VH and VL domains of SEQ ID NO:49 and SEQ ID NO:50. In some embodiments, the nucleic acid molecule encoding a CAR polypeptide comprises an scFV comprising an amino acid sequence having at least any of 80%, 85%, 90%, 95%, 97%, 98%, 99% or 99.5% of SEQ ID NO: 49 and SEQ ID NO: 50, provided the scFV domain binds to a citrullinated antigen as described herein. In some embodiments, the cell is a T-cell, a CD4 T-cell, a Treg cell, a CD8 alpha T-cell, CD8 beta T cell, T helper cell, granulocyte (neutrophils, basophils, eosinophils), megakaryocytes, monocyte, macrophage and a dendritic cell, or a T memory stem cell. In some embodiments, the cell is a Treg cell.

### VI. METHODS OF USE

T cells, and in particular, Treg cells that express the CARs disclosed herein are useful in the treatment of hidradenitis suppurativa. Methods of use comprise administering an effective amount a pharmaceutical composition of this disclosure to a subject in need thereof, e.g., a subject suffering from hidradenitis suppurativa.

As used herein, the term “subject” refers to an individual animal. The term “patient” as used herein refers to a subject under the care or supervision of a health care provider such as a doctor or nurse. Subjects include mammals, such as humans and non-human primates, such as monkeys, as well as dogs, cats, horses, bovines, rabbits, rats, mice, goats, pigs, and other mammalian species. Subjects can also include avians. A patient can be an individual that is seeking treatment, monitoring, adjustment or modification of an existing therapeutic regimen, etc. The term “hidradenitis suppurativa subject” refers to an individual that has been diagnosed with hidradenitis suppurativa. Hidradenitis suppurativa patients can include individuals that have not received treatment, are currently receiving treatment, have had treatment, and those that have discontinued treatment.

As used herein, the terms “effective amount,” “effective dose,” and “therapeutically effective amount,” refer to an amount of an agent that is sufficient to generate a desired response, such as reduce or eliminate a sign or symptom of a condition or ameliorate a disorder. In some examples, an “effective amount” is one that treats (including prophylaxis) one or more symptoms and/or underlying causes of any of a disorder or disease and/or prevents progression of a disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control.

The pharmaceutical composition can be administered by any suitable route, including but not limited to intravenous, subcutaneous, intramuscular or intraperitoneal routes. An example of administration of a pharmaceutical composition includes storing the composition at 10 mg/ml in sterile isotonic aqueous saline solution for injection at 4° C., and diluting it in either 100 ml or 200 ml 0.9% sodium chloride for injection prior to administration to the patient. The pharmaceutical composition is administered by intravenous infusion over the course of 1 hour at a dose of between 0.2 and 10 mg/kg. In other embodiments, the pharmaceutical composition is administered by intravenous infusion over a period of between 15 minutes and 2 hours. In still other embodiments, the administration procedure is via sub-cutaneous bolus injection.

The dose of the composition is chosen in order to provide effective therapy for the patient and is in the range of less than 0.1 mg/kg body weight to about 25 mg/kg body weight or in the range 1 mg-2 g per patient. In some cases, the dose is in the range 1-100 mg/kg, or approximately 50 mg-8000 mg/patient. The dose may be repeated at an appropriate frequency which may be in the range once per day to once every three months, depending on the pharmacokinetics of the composition (e.g., half-life of the composition in the circulation) and the pharmacodynamic response (e.g., the duration of the therapeutic effect of the composition). In some embodiments, the in vivo half-life of between about 7 and about 25 days and composition dosing is repeated between once per week and once every 3 months.

Administration can be periodic. Depending on the route of administration, the dose can be administered, e.g., once every 1, 3, 5, 7, 10, 14, 21, or 28 days or longer (e.g., once every 2, 3, 4, or 6 months). In some cases, administration is more frequent, e.g., 2 or 3 times per day. The patient can be monitored to adjust the dosage and frequency of administration depending on therapeutic progress and any adverse side effects, as will be recognized by one of skill in the art.

Thus, in some embodiments, additional administration is dependent on patient progress, e.g., the patient is monitored between administrations. For example, after the first administration or round of administrations, the patient can be monitored for rate of symptom relief.

In certain embodiments, the T cells described herein are administered to a skin lesion of subjects having hidradenitis suppurativa.

An exemplary method of this disclosure includes isolating T lymphocytes from a biological sample obtained from the subject. Such T cell can be isolated by immunoaffinity, for example, using solid supports derivatized and anti-CD4 antibodies. CD4+T regulatory cells (Treg) can be separated from non-Treg cells based on their marker profile. Treg cells are CD4+, CD25+, CD127lo. Non Treg cells are: CD4+, CD25+ and CD127+. The isolated Treg cells are then transfected with an expression vector encoding a chimeric antigen receptor (CAR) of this disclosure. The transfected cells are expanded. The expanded cells are administered to the subject.

### VII. KITS

As used herein, the term “kit” refers to a collection of items intended for use together. The kit can optionally include a reference agent and/or instructions for use thereof. A kit can further include a shipping container adapted to hold a container, such as a vial that contains a composition as disclosed herein. A kit can include a container that contains within it the collection of items.

Kits of this disclosure can comprise a pharmaceutical composition as described herein, contained in a container, such as a bag or bottle for intravenous administration. Also included in the kit can be a fluidic conduit, such as a plastic tube, with a drip chamber. The drip chamber can communicate through a fluidic conduit with an intravenous needle. The fluidic conduit also can comprise one or more Y-sites and a roller clamp.

### VIII. ENUMERATED EMBODIMENTS

1. A method of treating a subject suffering from hidradenitis suppurativa, comprising administering to the subject an effective amount of a pharmaceutical composition comprising a plurality of modified regulatory T (Treg) cells and a pharmaceutically acceptable excipient, wherein
the modified Treg cells express a chimeric antigen receptor (CAR) comprising an antigen-binding domain, a hinge domain, a transmembrane domain, one or more co-stimulatory domains, and an intracellular signaling domain,
the antigen binding domain specifically binds to one or more different citrullinated proteins or citrullinated fragments thereof, and
the modified Treg cells are CD4+, CD25+ and CD127lo.
2. The method of embodiment 1, wherein the antigen-binding domain of the CAR specifically binds to citrullinated vimentin.
3. The method of embodiment 1, wherein the antigen-binding domain of the CAR binds to all three of (i) citrullinated vimentin, (ii) citrullinated filaggrin, and (iii) citrullinated fibrinogen, or citrullinated peptides fragments thereof.
4. The method of embodiment 3, wherein the antigen-binding domain of the CAR comprises a VH domain and a VL domain, wherein:
   (i) the VH domain comprises a VH-CDR1 comprising the amino acid sequence of SEQ ID NO:32, a VH-CDR2 comprising the amino acid sequence of SEQ ID NO:34, and a VH-CDR3 comprising the amino acid sequence of SEQ ID NO:36, and
   (ii) the VL domain of the target-binding domain comprises a VL-CDR1 comprising the amino acid sequence of SEQ ID NO:39, a VL-CDR2 comprising the amino acid sequence of SEQ ID NO:41, and a VL-CDR3 comprising the amino acid sequence of SEQ ID NO:43.
5. The method of embodiment 3, wherein the antigen-binding domain of the CAR comprises a VH domain and a VL domain, wherein:
   (i) the VH domain comprises a VH-CDR1 comprising the amino acid sequence of SEQ ID NO:53, a VH-CDR2 comprising the amino acid sequence of SEQ ID NO:55, and a VH-CDR3 comprising the amino acid sequence of SEQ ID NO:57, and
   (ii) the VL domain of the target-binding domain comprises a VL-CDR1 comprising the amino acid sequence of SEQ ID NO:60, a VL-CDR2 comprising the amino acid sequence of SEQ ID NO:62, and a VL-CDR3 comprising the amino acid sequence of SEQ ID NO:64.
6. The method of any one of embodiments 1-5 wherein the intracellular signaling domain of the CAR is derived from CD3-zeta.
7. The method of any one of embodiments 1-6, wherein the at least one co-stimulatory domain of the CAR comprises a co-stimulatory domain of a member of the group consisting of FceR1g, Fcg, CD28, CD134 (OX40), CD137 (4-1BB), CTLA-4, CTLA-4/CD-28 hybrid, DAP10, CD27, 2B4, and combinations thereof, optionally wherein the at least one co-stimulatory domain comprises a CD28 and/or a 4-1 BB co-stimulatory domain.
8. The method of any one of embodiments 1-7, wherein the antigen-binding domain of the CAR comprises an antibody, an antibody fragment, a camelid nanobody, a heavy chain only antibody or an aptamer.
9. The method of any one of embodiments 1-8, wherein the transmembrane domain of the CAR is a CD8 transmembrane domain or a CD28 transmembrane domain.
10. The method of any one of embodiments 1-9, wherein the hinge domain of the CAR is a CD8 hinge domain or a CD28 hinge domain.
11. The method of any one of embodiments 1-10, wherein the CAR further comprising a signal peptide.
12. The method of embodiment 11, wherein the signal peptide of the CAR is a CD8 signal peptide or a GM-CSF signal peptide.
13. The method of any one of embodiments 1-12, wherein the antigen-binding domain of the CAR comprises a single chain fragment.
14. The method of embodiment 13, wherein the single chain fragment comprises a single chain variable fragment (scFv).
15. The method of Embodiment 14, wherein the scFv fragment comprises:
(a) a VH domain comprising the amino acid sequence of SEQ ID NO:1 or the amino acid sequence having at least 95% sequence identity to SEQ ID NO:1; and
(b) a VL domain comprising the amino acid sequence of SEQ ID NO:4 or the amino acid sequence having at least 95% sequence identity to SEQ ID NO:4.
16. The method of Embodiment 14, wherein the scFv fragment comprises:
(a) a VH domain comprising the amino acid sequence of SEQ ID NO:49 or the amino acid sequence having at least 95% sequence identity to SEQ ID NO:49; and
(b) a VL domain comprising the amino acid sequence of SEQ ID NO:50 or the amino acid sequence having at least 95% sequence identity to SEQ ID NO:50.
17. The method of Embodiment 14, wherein the scFv fragment comprises:
(a) a VH selected from:

(1) SBT01 VH (M)
(SEQ ID NO: 1)
HLHLQESGPGLVKPSETLSLTCTVSGGSINDTTYYWGWIRQPPGKGLEW
IGSIYYRGNTHYNSSLRSRVTMSVDTSKNRFSLKVTSVTAADTAVYYCA
RLDPFDYWGRGTLVTVSS;
or
(2) SBT01 VH (G)
(SEQ ID NO: 2)
QLQLQESGPGLVKPSETLSLTCTVSGGSISSSSYYWGWIRQPPGKGLEW
IGSIYYSGSTYYNPSLKSRVTISVDTSKNQFSLKLSSVTAADTAVYYCA
RLDPFDYWGRGTLVTVSS;

and
(b) a VL selected from:

(1) SBT01 VL (M)
(SEQ ID NO: 3)
SYVLTQPPSVSLAPGETATITCGGDDIENQNVNWYQQKSGQAPMLLIFF
DTRRPSGIPERFSGSRSEDTANLTITRVEAGDDADYFCQVYDRKTDHQV
FGPGTTVTVL
or
(2) SBT01 VL (G)
(SEQ ID NO: 4)
SYVLTQPPSVSVAPGKTARITCGGNNIGSKSVHWYQQKPGQAPVLVIYY
DSDRPSGIPERFSGSNSGNTATLTISRVEAGDEADYYCQVWDSSSDHQV
FGTGTKVTVL.

18. The method of any one of embodiments 1-17, wherein the VH-VL fragments are joined by a linker selected from the group consisting of (i) GGGSx3 (SEQ ID NO:20), (ii) Whitlow 218 (SEQ ID NO:21), and (iii) AB Pur (SEQ ID NO:22).
19. The method of embodiment 18, wherein the scFv comprises the amino acid sequence of:
(a) SBT01 G-VHVL-GGGSx3 Linker of SEQ ID NO:5; or
(b) SBT01 G-VHVL-Whitlow 218 Linker of SEQ ID NO:6; or
(c) SBT01 G-VHVL-AB pur Linker of SEQ ID NO:7; or
(d) U01-VHVL-AB pur Linker of SEQ ID NO:51.
20. The method of any one of embodiments 1-19, wherein the modified Treg cells are human T cells and the subject is a human patient.
21. The method of any one of embodiments 1-20, wherein the modified Treg cells are a primary T cells.
22. The method of any one of embodiments 1-21, wherein the pharmaceutical composition is administered intravenously.
23. A method of treating a subject suffering from hidradenitis suppurativa, the method comprising:
(a) isolating T cells from a biological sample obtained from the subject;
(b) enriching the T cells for T regulatory cells (Treg);
(c) transfecting the enriched Treg cells with an expression vector encoding a chimeric antigen receptor (CAR) comprising an antigen-binding domain, a hinge domain, a transmembrane domain, one or more co-stimulatory domains, and an intracellular signaling domain to produce modified Treg cells expressing the CAR, wherein
the antigen binding domain specifically binds to one or more different citrullinated proteins or citrullinated fragments thereof, and
the modified Treg cells are CD4+, CD25+ and CD127lo;
(d) expanding the modified Treg cells; and
(e) administering the modified Treg cells to the subject.
24. The method of embodiment 23, wherein the expansion comprises using anti-CD3/CD28 coated beads.
25. The method of embodiment 23, wherein the expansion does not comprise using anti-CD3/CD28 coasted beads.
26. The method of any one of embodiments 23-25, wherein the transfection occurs by use of a viral vector, electroporation, heat shock, bacteriophage, sonication, or calcium phosphate.
27. The method of any one of embodiments 23-26 further comprising administering one or more anti-inflammatory and/or therapeutic agents to the subject.
28. The method of embodiment 27, wherein the one or more anti-inflammatory agents comprises an antibody that inhibits a pro-inflammatory cytokine.
29. The method of embodiment 28, wherein the anti-inflammatory agents comprise an anti-TNF antibody, an anti-IL-6 antibody, or a combination thereof.
30. The method of any one of embodiments 23-29 and further comprising limitations of any one of embodiments 2-22.
31. Use of a plurality of modified regulatory T (Treg) cells in the manufacture of a medicament for treating hidradenitis suppurativa in a subject, wherein
the modified Treg cells express a chimeric antigen receptor (CAR) comprising an antigen-binding domain, a hinge domain, a transmembrane domain, one or more co-stimulatory domains, and an intracellular signaling domain,
the antigen binding domain specifically binds to one or more different citrullinated proteins or citrullinated fragments thereof, and
the modified Treg cells are CD4+, CD25+ and CD127lo.
32. The use of embodiment 31 and further comprising limitations of any one of embodiments 2-22.
33. A plurality of modified regulatory T (Treg) cells for use in a method of treating hidradenitis suppurativa in a subject, the method comprising administering to the subject the plurality of modified regulatory T (Treg) cells, wherein
the modified Treg cells express a chimeric antigen receptor (CAR) comprising an antigen-binding domain, a hinge domain, a transmembrane domain, one or more co-stimulatory domains, and an intracellular signaling domain,
the antigen binding domain specifically binds to one or more different citrullinated proteins or citrullinated fragments thereof, and
the modified Treg cells are CD4+, CD25+ and CD127lo.
34. The plurality of modified Treg cells for use of embodiment 33 and further comprising limitations of any one of embodiments 2-22.

### Examples

Abbreviations: ACPA (anti-citrullinated protein antibodies); CAR (chimeric antigen receptor); CF (citrullinated fibrinogen); Cit (citrullinated); CitP (citrullinated protein); CRP (C-reactive protein); CV (citrullinated vimentin); EGFR (epidermal growth factor receptor); HD (healthy donor); HS (hidradenitis suppurativa); IV (intravenous); LPS (lipopolysaccharide); PAD2 (peptidylarginine deiminase 2); PBMC (peripheral blood mononuclear cells); PBS (phosphate buffered saline); RA (rheumatoid arthritis); scFv (single-chain variable fragment); SF (synovial fluid); Teff (effector T cell); Treg (regulatory T cell); TSDR (Treg-specific de-methylation region); UTD (untransduced); and VICM (citrullinated and MMP-degraded vimentin).

### Example 1: Detection of Citrullinated Antigen at Sites of Inflammation of Hidradenitis Suppurativa and Rheumatoid Arthritis Patients

To evaluate the presence of citrullinated antigens in biopsy samples from HS patients, an immunohistochemistry (IHC) assay was developed. The IHC assay employed an anti-citrullinated vimentin antibody (anti-CV Ab), which contains the VH and VL of the single chain antigen-binding domain (scFV portion) of the S01 CAR and the Fc portion of a mouse IgG2a (S01-mIgG2a). The assay was optimized for use in fixed tissues using formalin-fixed paraffin-embedded positive and negative control cell lines. The S01-mIgG2a Ab produced positive staining on SKNBE2 cells overexpressing PAD2 and PAD4 (positive control cell lines) and did not react with the negative control cell line SKNBE2 WT. The specific reactions of the S01-mIgG2a Ab in all staining runs were accompanied by a negative control antibody where a lack of specific reactivity was demonstrated. To detect binding in patient biopsy samples, the S01-mIgG2a Ab was applied at a concentration of 12 μg/mL to sections of HS skin (n=58) and normal skin from adjacent samples (n=10), and then visualized using the EnVision FLEX system on the Dako Autostainer Link 48. Additional sections from all tested samples were stained with hematoxylin and eosin (H&E) to evaluate tissue quality, and to determine the extent of inflammatory cell infiltration. Additionally, all samples were stained with a negative control mouse IgG antibody to confirm specificity of the S01-mIgG2a Ab staining.

### Results

Strong reactivity to S01-mIgG2a Ab staining was observed in HS skin biopsy sections across multiple cell types, including squamous epithelial cells, synovial lining cells, and immune cells, as well as extracellular staining, especially in necrotic tissue areas. In contrast, little staining was observed in normal skin sections.

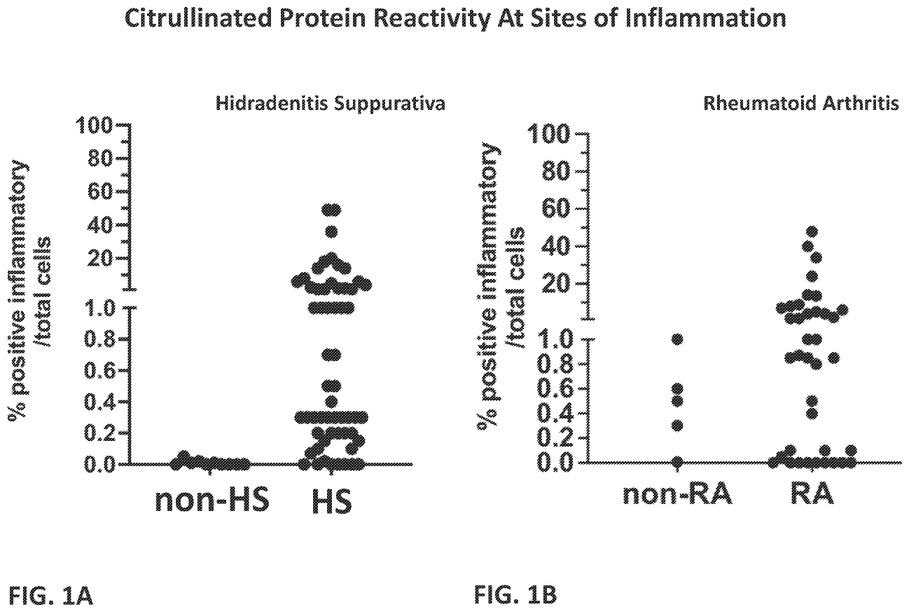

FIG. 1A-1B shows that citrullinated protein (CitP) is present in samples of hidradenitis suppurativa (HS) lesions and in synovial tissues samples from rheumatoid arthritis (RA) patients, but not in samples of normal skin (non-HS) and normal synovial tissue (non-RA).

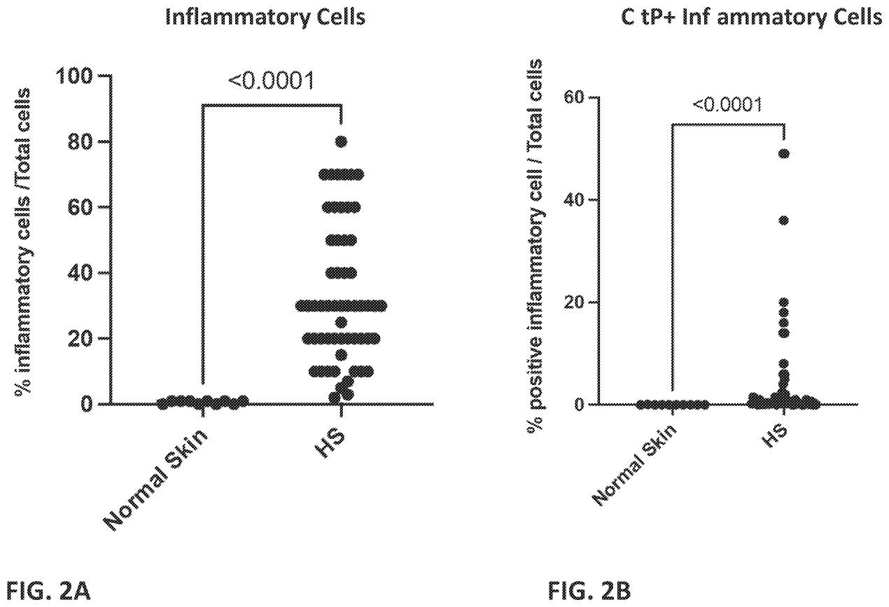

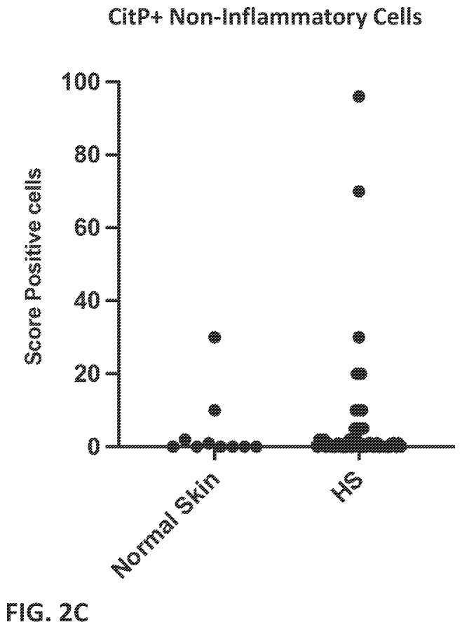

FIG. 2A-2B shows higher levels of inflammation, including higher numbers of CitP+ inflammatory cells in lesional HS skin as compared to adjacent normal skin. In addition, higher numbers of viable CitP+ non-inflammatory cells were present in lesional HS skin as compared to adjacent normal skin, as shown in FIG. 2C. Thus, immunohistochemical evaluation demonstrated that there is increased inflammation and increased target antigen (CitP) expression in lesional HS skin.

### Example 2: Detection of Citrullinated Antigen in Serum from Hidradenitis Suppurativa and Rheumatoid Arthritis Patients

To evaluate the presence of citrullinated protein (CitP) in serum from HS and RA patients a cell based assay and an ELISA were completed with CV-CAR transduced cells and an anti-CV-Ab, respectively. In addition, levels of VICM in serum were measured. Levels of c-reactive protein (CRP) and anti-citrullinated protein antibodies (ACPA) were measured in HS and normal serum samples.

Jurkat-FF-Luciferase transduction. About 24×10⁶ Jurkat-FF-luc cells were pelleted and resuspended in 24 ml RPMI containing protamine sulfate and virus at a MOI of 3, to express CV-CARs. Cells were mixed and 4 ml were aliquoted into each well of a 6-well plate. Plates were then spun and placed in an incubator overnight. Cells were pelleted and reseeded in 25 ml RPMT in T75 flasks.

Stimulus. Transduced cells were pelleted and resuspended in RPMI and placed in wells of black/white plates. Serum samples of HS patients and RA patients were thawed, vortexed, and diluted in RPMI before being added to plates containing transduced cells. Normal serum samples served as a control. Alternatively, citrullinated or non-citrullinated proteins were added to plates as stimuli. Cells were cultured in the presence of stimuli at 37° C.

Luciferase Assay. Approximately 24 hours later, 75 μl BioGlo reagent was added to each well, and the plates were incubated 2-3 mins in the dark. Luminescence was then read on a plate reader.

### Results

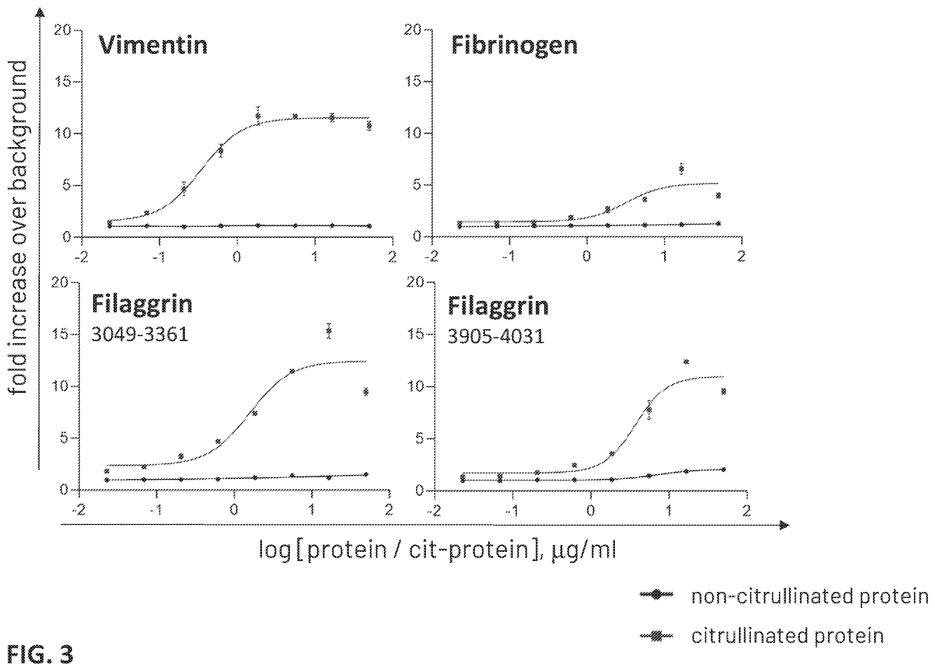

As shown in FIG. 3, the CV-CAR reporter cell line was activated by citrullinated vimentin, fibrinogen, and filaggrin proteins, but not by their non-citrullinated counterparts.

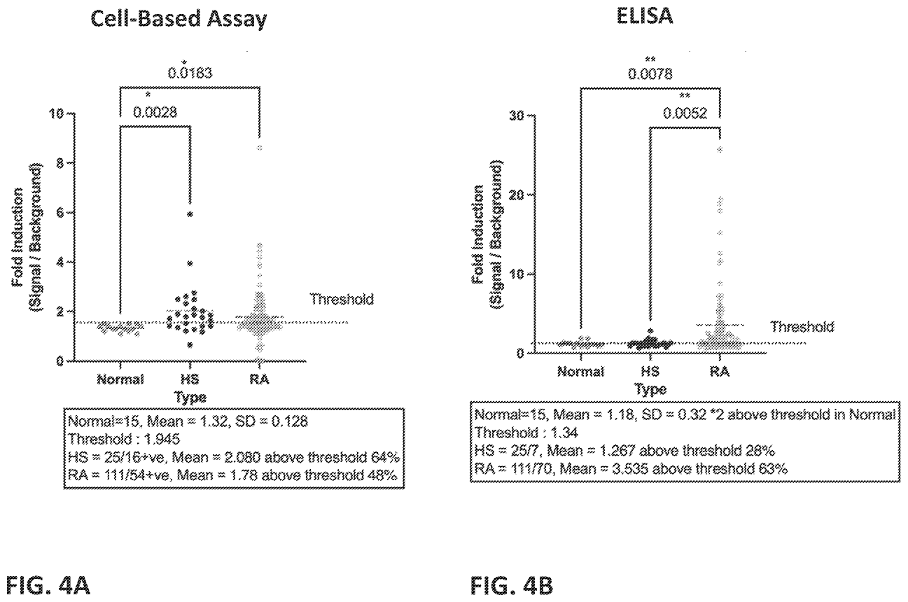

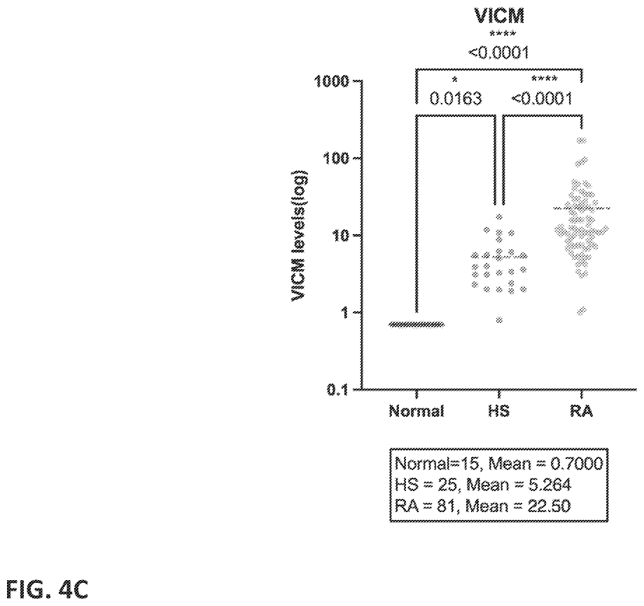

As shown in FIG. 4A-4B, levels of CitP are higher in serum from patients with HS or RA than in serum from normal control subjects. Likewise, as shown in FIG. 4C, higher levels of citrullinated and MMP-degraded vimentin (VICM) are present in serum from patients with HS or RA than in serum from normal control subjects.

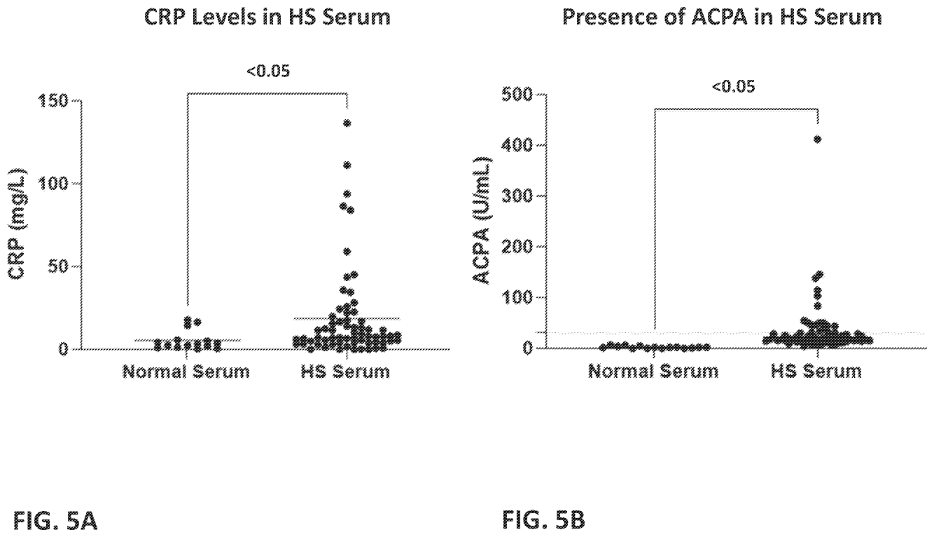

As shown in FIG. 5A-5B, serum samples of HS patients have elevated levels of CRP and ACPA as compared to normal serum samples.

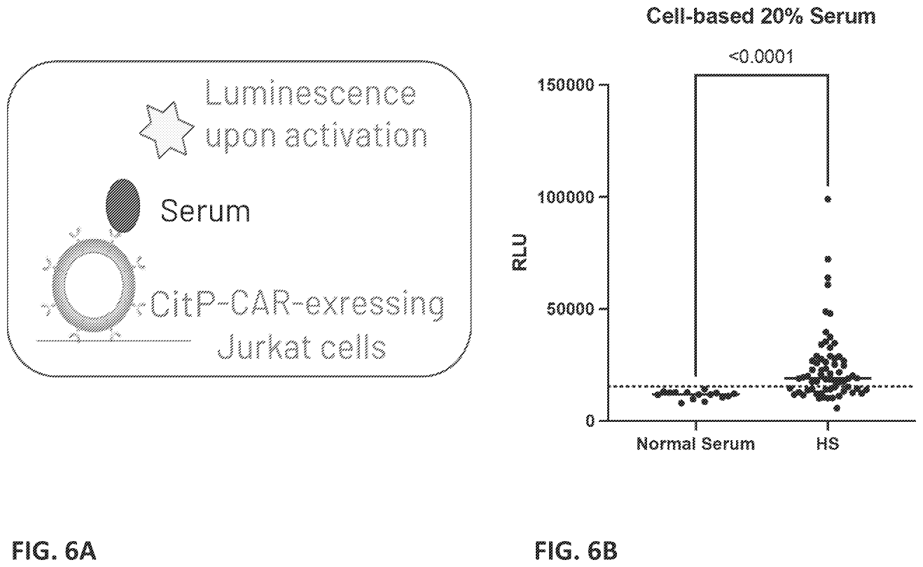

FIG. 6A provides a schematic overview of the cell-based detection assay used to measure the presence of citrullinated antigen in serum. FIG. 6B shows that serum samples of HS patients have elevated levels of citrullinated-antigen as compared to normal serum samples. Strikingly, 42 of 65 HS serum samples were found to have levels of Cit-P that were above the limit of detection shown by the dotted line.

### Example 3: Treg Cell Isolation and Characterization from Hidradenitis Suppurativa Patients and Healthy Donors

Treg cells recovered from whole blood samples obtained from healthy donors and HS patients were immunophenotyped.

Treg isolation. Peripheral blood mononuclear cells (PBMC) were isolated by density gradient centrifugation. Treg cells were next isolated using FACS by gating for CD4+CD25+CD127lo cells.

Flow Cytometry and FACS Analysis: Treg cell cultures were collected and centrifuged at 300×g for 5 min and then resuspended in 1× Flowstain Buffer (Invitrogen) with anti-Ki67, anti-CTLA4, and anti-HLA-DR surface staining antibodies. Treg cells were incubated for 30 min at 4° C., then centrifuged and washed with 1× Flow stain Buffer. Stained cells were fixed with CytoFix (BD Biosciences) then analyzed by flow cytometry.

### Results

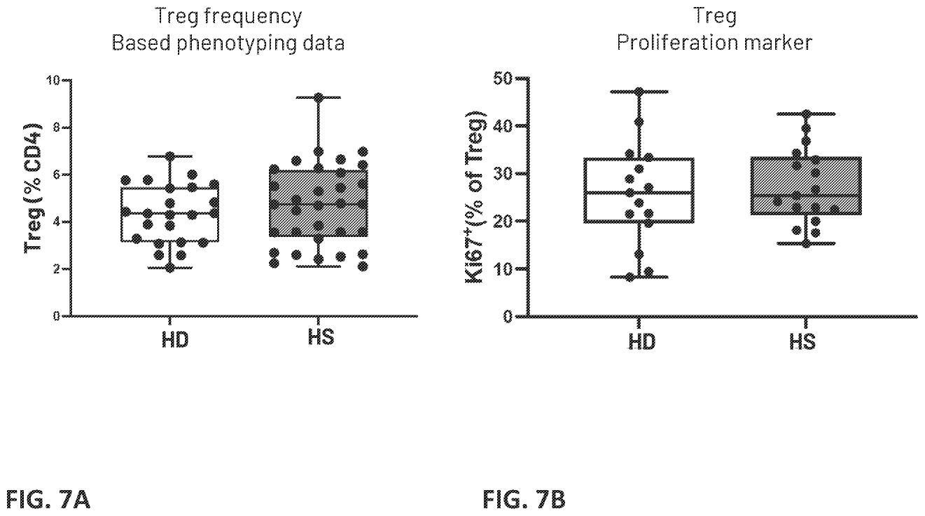

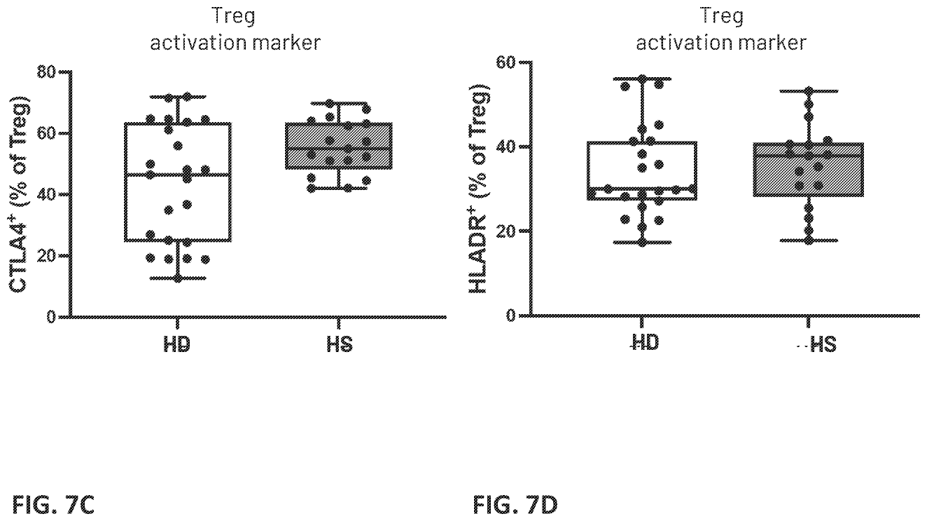

As shown in FIG. 7A-7D, HS patients have a comparable Treg frequency and phenotype as healthy donors. The CD4 and the CD8 T cell subset profiles of HS patients are similar to the profile of HD controls. While Tregs from HS patients have the same frequency as Tregs from healthy donors, Tregs from HS patients have higher absolute counts. This difference is driven by the higher pan CD4 T-cell frequency and counts.

### Example 4: Treg Cell Isolation, Transduction, Expansion and Characterization

Treg cells were recovered from whole blood samples obtained from healthy donors and HS patients.

Treg Isolation and Tissue Culture. Peripheral blood mononuclear cells (PBMC) were isolated by density gradient centrifugation. CD25+ cells were enriched by positive selection. Treg cells were isolated using FACS by gating for CD4+CD25+CD127lo cells. After isolation, expansions were done starting with 140,000 Treg cells in 48-well tissue culture plates.

Transduction of Primary Tregs: Primary Treg cells were transduced with CV-CAR constructs on day 2 of expansion via spin-occulation in the presence of protamine sulfate.

Flow Cytometry and FACS Analysis: On Day 14 of expansion, CV-CAR expressing Tregs were stained for transcription factors FoxP3 and Helios. Cells were fixed and permeabilized using eBiosciences FoxP3 transcription factor buffer set (eBiosciences).

### Results

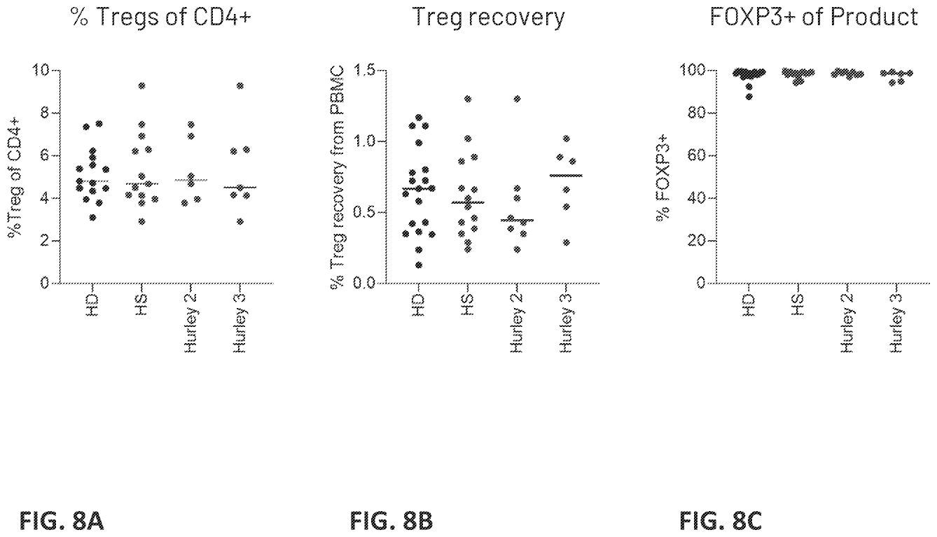

As shown in FIG. 8A-8B, Treg frequency and recovery during production of CV-CAR Treg cells are comparable for HS patients and HD. This observation holds true for patients with both moderate and severe HS (Hurley 2 and Hurley 3). CV-CAR Treg cells can be successfully generated from HS patients as evidenced by FOXP3 (FIG. 8C) and Helios (data not shown) expression.

### Example 5: Assessment of CV-CAR Treg Suppression Activity and Activation by Citrullinated Vimentin In Vitro

Treg cells were recovered from whole blood samples obtained from healthy donors (N=6) or HS patients (N=7).

Treg Isolation and Tissue Culture. Peripheral blood mononuclear cells (PBMC) were isolated by density gradient centrifugation. CD25+ cells were enriched by positive selection. Treg cells were next isolated using FACS by gating for CD4+CD25+CD127lo cells. After isolation, expansions were done starting with 140,000 Treg cells in 48-well tissue culture plates.

Transduction of Primary Tregs: Primary Treg cells were transduced with CV-CAR constructs on day 2 of expansion via spin-occulation in the presence of protamine sulfate.

Flow Cytometry and FACS Analysis: Treg cell cultures were collected and centrifuged at 300×g for 5 min and then resuspended in 1× Flowstain Buffer (Invitrogen) with a viability dye (Invitrogen), anti-EGFR and CD71 surface staining antibody. Treg cells were incubated for 30 min at 4° C., then centrifuged and washed with 1× Flow stain Buffer. Stained cells were fixed with CytoFix (BD Biosciences) then analyzed by flow cytometry. Expression of Helios and FoxP3 by Treg cells was also assessed by flow cytometry.

Activation of Tregs: Untransduced and CV-CAR-expressing Treg cells were cultured in vitro with the citrullinated vimentin beads ranging in dose from 1:243 to 1:1 pCVbead:Treg ratio. Treg activation was assessed by measurement of percentages of proliferating cells and CD71 expression.

Treg suppression assay: Tregs were co-cultured with either CD3/CD28-activated or allo total T cells to assess Treg suppression of target cells. Teff cells were co-cultured at the following Treg:Tresp ratios of 1:32, 1:16, 1:4, 1:2, or 1:1.

### Results

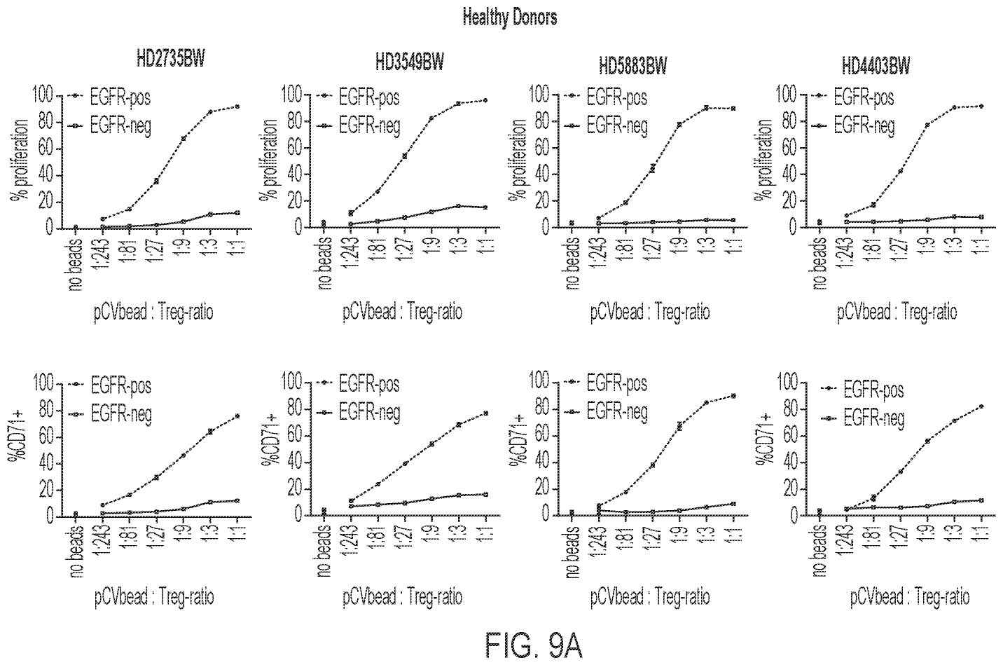

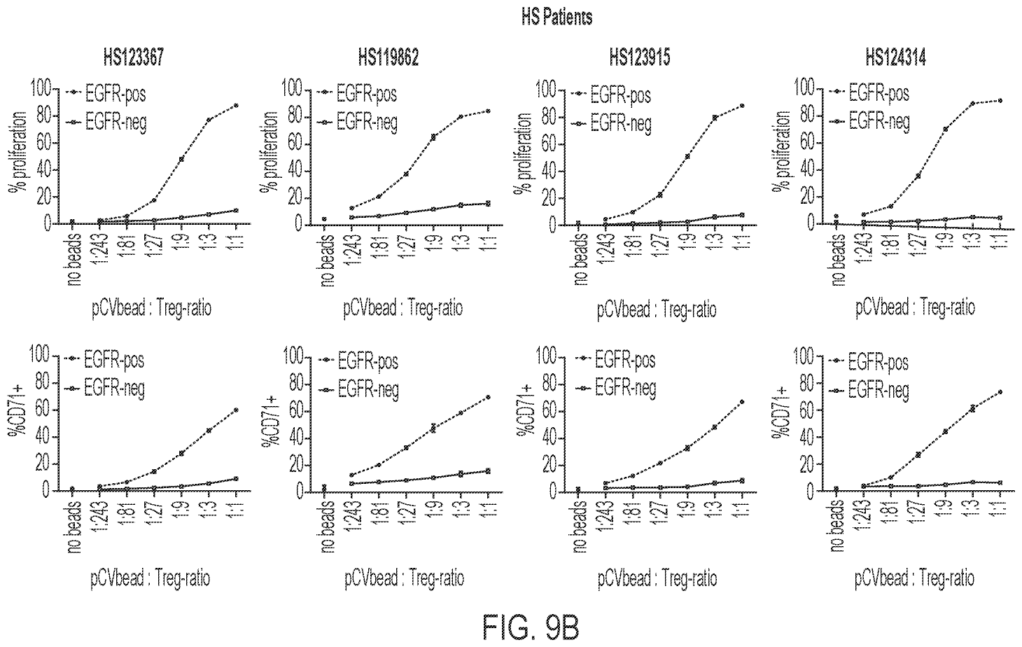

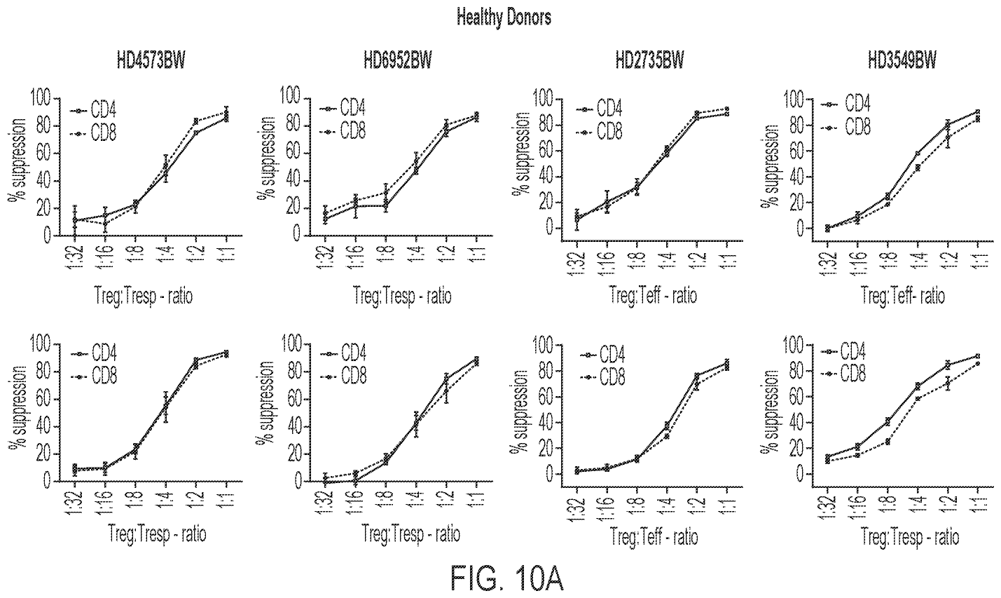

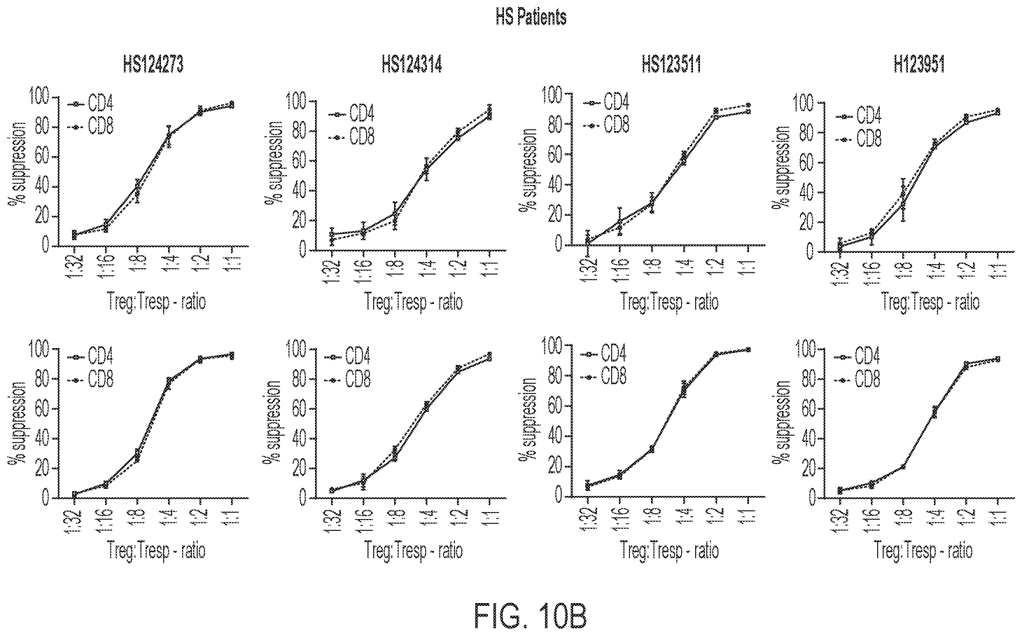

No disease-specific differences in Treg cell isolation, phenotype or function have been observed to date, Tregs from healthy donors and HS patients have similar levels of Helios and FoxP3 (data not shown). FIG. 9A-9B and FIG. 10A-10B show that Tregs from healthy donors and HS patients have similar capacity for proliferation and activation by CAR stimuli (pCV-beads) and similar suppressive activity.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

We claim:

1. A method of treating a subject suffering from hidradenitis suppurativa, comprising administering to the subject an effective amount of a pharmaceutical composition comprising a plurality of modified regulatory T (Treg) cells and a pharmaceutically acceptable excipient, wherein
the modified Treg cells express a chimeric antigen receptor (CAR) comprising an antigen-binding domain, a hinge domain, a transmembrane domain, one or more co-stimulatory domains, and an intracellular signaling domain,
the antigen binding domain specifically binds to one or more different citrullinated proteins or citrullinated fragments thereof, and
the modified Treg cells are CD4+, CD25+ and CD127lo.

2. The method of claim 1, wherein the antigen-binding domain of the CAR specifically binds to citrullinated vimentin.

3. The method of claim 1, wherein the antigen-binding domain of the CAR binds to all three of (i) citrullinated vimentin, (ii) citrullinated filaggrin, and (iii) citrullinated fibrinogen, or citrullinated peptides fragments thereof.

4. The method of claim 3, wherein the antigen-binding domain of the CAR comprises a VH domain and a VL domain, wherein:
(i) the VH domain comprises a VH-CDR1 comprising the amino acid sequence of SEQ ID NO:32, a VH-CDR2 comprising the amino acid sequence of SEQ ID NO:34, and a VH-CDR3 comprising the amino acid sequence of SEQ ID NO:36, and
(ii) the VL domain of the target-binding domain comprises a VL-CDR1 comprising the amino acid sequence of SEQ ID NO:39, a VL-CDR2 comprising the amino acid sequence of SEQ ID NO:41, and a VL-CDR3 comprising the amino acid sequence of SEQ ID NO:43.

5. The method of claim 3, wherein the antigen-binding domain of the CAR comprises a VH domain and a VL domain, wherein:
(i) the VH domain comprises a VH-CDR1 comprising the amino acid sequence of SEQ ID NO:53, a VH-CDR2 comprising the amino acid sequence of SEQ ID NO:55, and a VH-CDR3 comprising the amino acid sequence of SEQ ID NO:57, and
(ii) the VL domain of the target-binding domain comprises a VL-CDR1 comprising the amino acid sequence of SEQ ID NO:60, a VL-CDR2 comprising the amino acid sequence of SEQ ID NO:62, and a VL-CDR3 comprising the amino acid sequence of SEQ ID NO:64.

6. The method of claim 1, wherein the intracellular signaling domain of the CAR is derived from CD3-zeta.

7. The method of claim 1, wherein the at least one co-stimulatory domain of the CAR comprises a co-stimulatory domain of a member of the group consisting of FceR1 g, Fcg, CD28, CD134 (OX40), CD137 (4-1BB), CTLA-4, CTLA-4/CD-28 hybrid, DAP10, CD27, 2B4, and combinations thereof.

8. The method of claim 1, wherein the antigen-binding domain of the CAR comprises an antibody, an antibody fragment, a camelid nanobody, a heavy chain only antibody or an aptamer.

9. The method of claim 1, wherein the transmembrane domain of the CAR is a CD8 transmembrane domain or a CD28 transmembrane domain.

10. The method of claim 1, wherein the hinge domain of the CAR is a CD8 hinge domain or a CD28 hinge domain.

11. The method of claim 1, wherein the CAR further comprising a signal peptide.

12. The method of claim 11, wherein the signal peptide of the CAR is a CD8 signal peptide or a GM-CSF signal peptide.

13. The method of claim 1, wherein the antigen-binding domain of the CAR comprises a single chain fragment.

14. The method of claim 13, wherein the single chain fragment comprises a single chain variable fragment (scFv).

15. The method of claim 14, wherein the scFv fragment comprises:
(a) a VH domain comprising the amino acid sequence of SEQ ID NO:1 or the amino acid sequence having at least 95% sequence identity to SEQ ID NO:1; and
(b) a VL domain comprising the amino acid sequence of SEQ ID NO:4 or the amino acid sequence having at least 95% sequence identity to SEQ ID NO:4.

16. The method of claim 14, wherein the scFv fragment comprises:
(a) a VH domain comprising the amino acid sequence of SEQ ID NO:49 or the amino acid sequence having at least 95% sequence identity to SEQ ID NO:49; and
(b) a VL domain comprising the amino acid sequence of SEQ ID NO:50 or the amino acid sequence having at least 95% sequence identity to SEQ ID NO:50.

17. The method of claim 14, wherein the scFv fragment comprises:
(a) a VH selected from:
(1) SBT01 VH (M)
(SEQ ID NO: 1)
HLHLQESGPGLVKPSETLSLTCTVSGGSINDTTYYWGWIRQPPGKGLEW
IGSIYYRGNTHYNSSLRSRVTMSVDTSKNRFSLKVTSVTAADTAVYYCA
RLDPFDYWGRGTLVTVSS;
or
(2) SBT01 VH (G)
(SEQ ID NO: 2)
QLQLQESGPGLVKPSETLSLTCTVSGGSISSSSYYWGWIRQPPGKGLEW
IGSIYYSGSTYYNPSLKSRVTISVDTSKNQFSLKLSSVTAADTAVYYCA
RLDPFDYWGRGTLVTVSS;
and
(b) a VL selected from:
(1) SBT01 VL (M)
(SEQ ID NO: 3)
SYVLTQPPSVSLAPGETATITCGGDDIENQNVNWYQQKSGQAPMLLIFF
DTRRPSGIPERFSGSRSEDTANLTITRVEAGDDADYFCQVYDRKTDHQV
FGPGTTVTVL
or
(2) SBT01 VL (G)
(SEQ ID NO: 4)
SYVLTQPPSVSVAPGKTARITCGGNNIGSKSVHWYQQKPGQAPVLVIYY
DSDRPSGIPERFSGSNSGNTATLTISRVEAGDEADYYCQVWDSSSDHQV
FGTGTKVTVL.

18. The method of claim 17, wherein the VH-VL fragments are joined by a linker selected from the group consisting of (i) GGGSx3 (SEQ ID NO:20), (ii) Whitlow 218 (SEQ ID NO:21), and (iii) AB Pur (SEQ ID NO:22).

19. The method of claim 18, wherein the scFv comprises the amino acid sequence of:
(a) SBT01 G-VHVL-GGGSx3 Linker of SEQ ID NO:5; or
(b) SBT01 G-VHVL-Whitlow 218 Linker of SEQ ID NO:6; or
(c) SBT01 G-VHVL-AB pur Linker of SEQ ID NO:7; or
(d) U01-VHVL-AB pur Linker of SEQ ID NO:51.

20. The method of claim 1, wherein the modified Treg cells are human T cells and the subject is a human patient.

21. The method of claim 20, wherein the modified Treg cells are a primary T cells.

22. The method of claim 21, wherein the pharmaceutical composition is administered intravenously.

23. A method of treating a subject suffering from hidradenitis suppurativa, the method comprising:
(a) isolating T cells from a biological sample obtained from the subject;
(b) enriching the T cells for T regulatory cells (Treg);
(c) transfecting the enriched Treg cells with an expression vector encoding a chimeric antigen receptor (CAR) comprising an antigen-binding domain, a hinge domain, a transmembrane domain, one or more co-stimulatory domains, and an intracellular signaling domain to produce modified Treg cells expressing the CAR, wherein
the antigen binding domain specifically binds to one or more different citrullinated proteins or citrullinated fragments thereof, and
the modified Treg cells are CD4+, CD25+ and CD127lo;
(d) expanding the modified Treg cells; and
(e) administering the modified Treg cells to the subject.

24. The method of claim 23, wherein the expansion comprises using anti-CD3/CD28 coated beads.

25. The method of claim 23, wherein the expansion does not comprise using anti-CD3/CD28 coasted beads.

26. The method of claim 23, wherein the transfection occurs by use of a viral vector, electroporation, heat shock, bacteriophage, sonication, or calcium phosphate.

27. The method of claim 23, further comprising administering one or more anti-inflammatory and/or therapeutic agents to the subject.

28. The method of claim 27, wherein the one or more anti-inflammatory agents comprises an antibody that inhibits a pro-inflammatory cytokine.

29. The method of claim 28, wherein the anti-inflammatory agents comprise an anti-TNF antibody, an anti-IL-6 antibody, or a combination thereof.

30. The method of any one of claims 23-29 and further comprising limitations of any one of claims 2-22.

31. Use of a plurality of modified regulatory T (Treg) cells in the manufacture of a medicament for treating hidradenitis suppurativa in a subject, wherein
the modified Treg cells express a chimeric antigen receptor (CAR) comprising an antigen-binding domain, a hinge domain, a transmembrane domain, one or more co-stimulatory domains, and an intracellular signaling domain,
the antigen binding domain specifically binds to one or more different citrullinated proteins or citrullinated fragments thereof, and
the modified Treg cells are CD4+, CD25+ and CD127lo.

32. The use of embodiment 31 and further comprising limitations of any one of claims 2-22.

33. A plurality of modified regulatory T (Treg) cells for use in a method of treating hidradenitis suppurativa in a subject, the method comprising administering to the subject the plurality of modified regulatory T (Treg) cells, wherein
the modified Treg cells express a chimeric antigen receptor (CAR) comprising an antigen-binding domain, a hinge domain, a transmembrane domain, one or more co-stimulatory domains, and an intracellular signaling domain,
the antigen binding domain specifically binds to one or more different citrullinated proteins or citrullinated fragments thereof, and
the modified Treg cells are CD4+, CD25+ and CD127lo.

34. The plurality of modified Treg cells for use of embodiment 33 and further comprising limitations of any one of claims 2-22.


SEQUENCE LISTING (65 sequences)

SEQ ID NO: 1 - protein, 116 aa, Homo sapiens
HLHLQESGPGLVKPSETLSLTCTVSGGSINDTTYYWGWIRQPPGKGLEWIGSIYYRGNTHYNSSLRSRVTMSVDTSKNRFSLKVTSVTAADTAVYYCARLDPFDYWGRGTLVTVSS

SEQ ID NO: 2 - protein, 116 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARLDPFDYWGRGTLVTVSS

SEQ ID NO: 3 - protein, 108 aa, Homo sapiens
SYVLTQPPSVSLAPGETATITCGGDDIENQNVNWYQQKSGQAPMLLIFFDTRRPSGIPERFSGSRSEDTANLTITRVEAGDDADYFCQVYDRKTDHQVFGPGTTVTVL

SEQ ID NO: 4 - protein, 108 aa, Homo sapiens
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDHQVFGTGTKVTVL

SEQ ID NO: 5 - protein, 240 aa, synthetic construct
HLHLQESGPGLVKPSETLSLTCTVSGGSINDTTYYWGWIRQPPGKGLEWIGSIYYRGNTHYNSSLRSRVTMSVDTSKNRFSLKVTSVTAADTAVYYCARLDPFDYWGRGTLVTVSSGGGGSGGGGSGGGGSSYVLTQPPSVSVAPGKTARITCGGNNIGSKSVHWYQQKPGQAPVLVIYYDSDRPSGIPERFSGSNSGNTATLTISRVEAGDEADYYCQVWDSSSDHQVFGTGTKVTVLR

SEQ ID NO: 6 - protein, 243 aa, synthetic construct
HLHLQESGPGLVKPSETLSLTCTVSGGSINDTTYYWGWIRQPPGKGLEWIGSIYYRGNTHYNSSLRSRVTMSVDTSKNRFSLKVTSVTAADTAVYYCARLDPFDYWGRGTLVTVSSGSTSGSGKPGSGEGSTKGSYVLTQPPSVSVAPGKTARITCGGNNIGSKSVHWYQQKPGQAPVLVIYYDSDRPSGIPERFSGSNSGNTATLTISRVEAGDEADYYCQVWDSSSDHQVFGTGTKVTVLR

SEQ ID NO: 7 - protein, 249 aa, synthetic construct
HLHLQESGPGLVKPSETLSLTCTVSGGSINDTTYYWGWIRQPPGKGLEWIGSIYYRGNTHYNSSLRSRVTMSVDTSKNRFSLKVTSVTAADTAVYYCARLDPFDYWGRGTLVTVSSASSGGSTSGSGKPGSGEGSSGSARSYVLTQPPSVSVAPGKTARITCGGNNIGSKSVHWYQQKPGQAPVLVIYYDSDRPSGIPERFSGSNSGNTATLTISRVEAGDEADYYCQVWDSSSDHQVFGTGTKVTVLR

SEQ ID NO: 8 - DNA, 348 bp, Homo sapiens
  source 1..348: mol_type = unassigned DNA
cacctgcacttgcaggagtcgggcccaggacttgtgaagccttcggagaccctgtccctcacctgcactgtctctggtggctccatcaacgataccacttactactggggctggattcgccagccccccgggaagggactggagtggattgggagtatctattaccgggggaacacccactacaattcgtccctgaggagtcgcgtcaccatgtctgtcgacacttccaagaaccgattctccctgaaggtcacttctgtgactgccgcagacacggctgtctattactgtgcgagactcgacccatttgactactggggccgtggcaccctggtcactgtctcgagc

SEQ ID NO: 9 - DNA, 348 bp, Homo sapiens
  source 1..348: mol_type = unassigned DNA
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagactcgacccatttgactactggggccgtggcaccctggtcactgtctcgagc

SEQ ID NO: 10 - DNA, 324 bp, Homo sapiens
  source 1..324: mol_type = unassigned DNA
tcctatgtcctgactcagccaccctcagtgtcgctggccccgggagagacggccacaattacttgtggtggagacgacattgaaaatcaaaatgtcaactggtatcagcagaagtcaggtcaggcccctatgctgctcatcttctttgataccagacggccctcagggatcccggagcgattctctggctccaggtctgaggacacggccaacctgaccatcaccagggtcgaggccggggatgacgccgactatttctgtcaggtgtatgataggaagactgatcaccaagtcttcggacctgggaccacggtcaccgtccta

SEQ ID NO: 11 - DNA, 324 bp, Homo sapiens
  source 1..324: mol_type = unassigned DNA
tcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggacagtagtagtgatcaccaagtcttcggaactgggaccaaggtcaccgtccta

SEQ ID NO: 12 - DNA, 720 bp, synthetic construct
  source 1..720: mol_type = other DNA
cacctgcacttgcaggagtcgggcccaggacttgtgaagccttcggagaccctgtccctcacctgcactgtctctggtggctccatcaacgataccacttactactggggctggattcgccagccccccgggaagggactggagtggattgggagtatctattaccgggggaacacccactacaattcgtccctgaggagtcgcgtcaccatgtctgtcgacacttccaagaaccgattctccctgaaggtcacttctgtgactgccgcagacacggctgtctattactgtgcgagactcgacccatttgactactggggccgtggcaccctggtcactgtctcgagcggtggcggtggctcgggcggtggtgggtcgggtggcggcggatcttcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggacagtagtagtgatcaccaagtcttcggaactgggaccaaggtcaccgtcctacgc

SEQ ID NO: 13 - DNA, 729 bp, synthetic construct
  source 1..729: mol_type = other DNA
cacctgcacttgcaggagtcgggcccaggacttgtgaagccttcggagaccctgtccctcacctgcactgtctctggtggctccatcaacgataccacttactactggggctggattcgccagccccccgggaagggactggagtggattgggagtatctattaccgggggaacacccactacaattcgtccctgaggagtcgcgtcaccatgtctgtcgacacttccaagaaccgattctccctgaaggtcacttctgtgactgccgcagacacggctgtctattactgtgcgagactcgacccatttgactactggggccgtggcaccctggtcactgtctcgagcggaagcacgagtggttcaggcaaaccgggttccggtgaaggttcaacaaaaggttcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggacagtagtagtgatcaccaagtcttcggaactgggaccaaggtcaccgtcctacgc

SEQ ID NO: 14 - DNA, 747 bp, synthetic construct
  source 1..747: mol_type = other DNA
cacctgcacttgcaggagtcgggcccaggacttgtgaagccttcggagaccctgtccctcacctgcactgtctctggtggctccatcaacgataccacttactactggggctggattcgccagccccccgggaagggactggagtggattgggagtatctattaccgggggaacacccactacaattcgtccctgaggagtcgcgtcaccatgtctgtcgacacttccaagaaccgattctccctgaaggtcacttctgtgactgccgcagacacggctgtctattactgtgcgagactcgacccatttgactactggggccgtggcaccctggtcactgtctcgagcgcctctagcggggggagcacatcaggaagcggcaagcccggtagcggcgaaggctcctctggcagcgcccgctcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggacagtagtagtgatcaccaagtcttcggaactgggaccaaggtcaccgtcctacgc

SEQ ID NO: 15 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 16 - protein, 27 aa, Homo sapiens
FWVLVVVGGVLACYSLLVTVAFIIFWV

SEQ ID NO: 17 - protein, 24 aa, Homo sapiens
IYIWAPLAGTCGVLLLSLVITLYC

SEQ ID NO: 18 - protein, 22 aa, Homo sapiens
MLLLVTSLLLCELPHPAFLLIP

SEQ ID NO: 19 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 15 aa, synthetic construct
GGGGSGGGGSGGGGS

SEQ ID NO: 21 - protein, 18 aa, synthetic construct
GSTSGSGKPGSGEGSTKG

SEQ ID NO: 22 - protein, 24 aa, synthetic construct
ASSGGSTSGSGKPGSGEGSSGSAR

SEQ ID NO: 23 - protein, 20 aa, Homo sapiens
MALPVTALLLPLALLLHAAR

SEQ ID NO: 24 - protein, 16 aa, Homo sapiens
  MOD_RES 3: note = X = citrulline
  MOD_RES 11..12: note = X = citrulline
STXSVSSSSYXXMFGG

SEQ ID NO: 25 - protein, 15 aa, Homo sapiens
  MOD_RES 5: note = X = citrulline
  MOD_RES 10: note = X = citrulline
  MOD_RES 12: note = X = citrulline
VYATXSSAVXLXSSV

SEQ ID NO: 26 - protein, 15 aa, Homo sapiens
  MOD_RES 1: note = X = citrulline
  MOD_RES 13: note = X = citrulline
  MOD_RES 15: note = X = citrulline
XPAPPPISGGGYXAX

SEQ ID NO: 27 - protein, 19 aa, Homo sapiens
  MOD_RES 7: note = X = citrulline
SHQESTXGRSRGRSGRSGS

SEQ ID NO: 28 - protein, 42 aa, Homo sapiens
KRGRKKLLYIFKQPFMRPVQTTQEEDGCSCRFPEEEEGGCEL

SEQ ID NO: 29 - protein, 41 aa, Homo sapiens
RSKRSRLLHSDYMNMTPRRPGPTRKHYQPYAPPRDFAAYRS

SEQ ID NO: 30 - protein, 39 aa, Homo sapiens
IEVMYPPPYLDNEKSNGTIIHVKGKHLCPSPLFPGPSKP

SEQ ID NO: 31 - protein, 30 aa, Homo sapiens
HLHLQESGPGLVKPSETLSLTCTVSGGSIN

SEQ ID NO: 32 - protein, 7 aa, Homo sapiens
DTTYYWG

SEQ ID NO: 33 - protein, 14 aa, Homo sapiens
WIRQPPGKGLEWIG

SEQ ID NO: 34 - protein, 16 aa, Homo sapiens
SIYYRGNTHYNSSLRS

SEQ ID NO: 35 - protein, 32 aa, Homo sapiens
RVTMSVDTSKNRFSLKVTSVTAADTAVYYCAR

SEQ ID NO: 36 - protein, 6 aa, Homo sapiens
LDPFDY

SEQ ID NO: 37 - protein, 11 aa, Homo sapiens
WGRGTLVTVSS

SEQ ID NO: 38 - protein, 22 aa, Homo sapiens
SYVLTQPPSVSVAPGKTARITC

SEQ ID NO: 39 - protein, 11 aa, Homo sapiens
GGNNIGSKSVH

SEQ ID NO: 40 - protein, 15 aa, Homo sapiens
WYQQKPGQAPVLVIY

SEQ ID NO: 41 - protein, 7 aa, Homo sapiens
YDSDRPS

SEQ ID NO: 42 - protein, 32 aa, Homo sapiens
GIPERFSGSNSGNTATLTISRVEAGDEADYYC

SEQ ID NO: 43 - protein, 11 aa, Homo sapiens
QVWDSSSDHQV

SEQ ID NO: 44 - protein, 11 aa, Homo sapiens
FGTGTKVTVLR

SEQ ID NO: 45 - protein, 16 aa, Homo sapiens
STRSVSSSSYRRMFGG

SEQ ID NO: 46 - protein, 15 aa, Homo sapiens
VYATRSSAVRLRSSV

SEQ ID NO: 47 - protein, 15 aa, Homo sapiens
RPAPPPISGGGYRAR

SEQ ID NO: 48 - protein, 19 aa, Homo sapiens
SHQESTRGRSRGRSGRSGS

SEQ ID NO: 49 - protein, 119 aa, Homo sapiens
EVKLIESGGGLVEPGRSLRLACTTSGFTFADYGLSWFRQGPGKGLEWVGFTGPKHLGETTECAPSVEDRCTISRDDSKSTVYLQMHRLQHEDTAVYFCVGPWFGDLLMWGQGTLVTVSS

SEQ ID NO: 50 - protein, 111 aa, Homo sapiens
AIQMTQSPSSLSASVGDRVSITCRATQDISTSLGWYHQRPGKAPRLLIYGASKVQTGVPSRFSGNGSGTEFTLTISSLQPEDIGTYYCLQDDGFPFTVGQGTKLDIKRAAA

SEQ ID NO: 51 - protein, 254 aa, synthetic construct
EVKLIESGGGLVEPGRSLRLACTTSGFTFADYGLSWFRQGPGKGLEWVGFTGPKHLGETTECAPSVEDRCTISRDDSKSTVYLQMHRLQHEDTAVYFCVGPWFGDLLMWGQGTLVTVSSASSGGSTSGSGKPGSGEGSSGSARAIQMTQSPSSLSASVGDRVSITCRATQDISTSLGWYHQRPGKAPRLLIYGASKVQTGVPSRFSGNGSGTEFTLTISSLQPEDIGTYYCLQDDGFPFTVGQGTKLDIKRAAA

SEQ ID NO: 52 - protein, 30 aa, Homo sapiens
EVKLIESGGGLVEPGRSLRLACTTSGFTFA

SEQ ID NO: 53 - protein, 5 aa, Homo sapiens
DYGLS

SEQ ID NO: 54 - protein, 14 aa, Homo sapiens
WFRQGPGKGLEWVG

SEQ ID NO: 55 - protein, 19 aa, Homo sapiens
FTGPKHLGETTECAPSVED

SEQ ID NO: 56 - protein, 32 aa, Homo sapiens
RCTISRDDSKSTVYLQMHRLQHEDTAVYFCVG

SEQ ID NO: 57 - protein, 8 aa, Homo sapiens
PWFGDLLM

SEQ ID NO: 58 - protein, 11 aa, Homo sapiens
WGQGTLVTVSS

SEQ ID NO: 59 - protein, 23 aa, Homo sapiens
AIQMTQSPSSLSASVGDRVSITC

SEQ ID NO: 60 - protein, 11 aa, Homo sapiens
RATQDISTSLG

SEQ ID NO: 61 - protein, 15 aa, Homo sapiens
WYHQRPGKAPRLLIY

SEQ ID NO: 62 - protein, 7 aa, Homo sapiens
GASKVQT

SEQ ID NO: 63 - protein, 32 aa, Homo sapiens
GVPSRFSGNGSGTEFTLTISSLQPEDIGTYYC

SEQ ID NO: 64 - protein, 9 aa, Homo sapiens
LQDDGFPFT

SEQ ID NO: 65 - protein, 13 aa, Homo sapiens
VGQGTKLDIKRAA